(19) United States
(12) Patent Application Publication
(10) Pub. No.: US 2006/0281690 A1
(43) Pub. Date: Dec. 14, 2006

(54) Crystalline (2E,4S)-4-[(N-{[(2R)-1-isopropylpiperidin-2-yI]-carbonyl}-3-methyl-L-valyl)(methyl)amino]-2,5-dimethylhex-2-enoic acid and its pharmaceutical uses

(75) Inventors: Silvio Campagna, Candia, NH (US); Mihaela Maria Pop, Amsterdam (NL)

Correspondence Address: NIXON PEABODY, LLP, 401 9TH STREET, NW, SUITE 900, WASHINGTON, DC, 20004-2128, US

(73) Assignee: Eisai Co. Ltd., Tokyo (JP)

(21) Appl. No.: 11/418,110

(22) Filed: May 5, 2006

Related U.S. Application Data
(60) Provisional application No. 60/677,811, filed on May 5, 2005.

(51) Int. Cl. A61K 38/00

(52) U.S. Cl. 514/19

(57) ABSTRACT

The invention relates to unsolvated and host-guest solvated crystalline forms of (2E,4S)-4-[(N-{[(2R)-1-isopropylpiperidin-2-yl]-carbonyl}-3-methyl-L-valyl)(methyl)amino]-2,5-dimethylhex-2-enoic acid, E7974, and their therapeutic uses. Pharmaceutical compositions containing crystalline forms of E7974 and a pharmaceutically acceptable carrier represent one embodiment of the invention. The invention also relates to methods for treating cancer, an inflammatory disorder, an autoimmune disorder, or a proliferative disorder as well as restenosis of blood vessels comprising the step of administering to a patient in need thereof a therapeutically effective amount of crystalline E7974.

### CROSS-REFERENCE TO RELATED APPLICATION

This application claims priority under 35 U.S.C. § 119 to provisional application Ser. No. 60/677,811, which is incorporated herein by reference.

### FIELD OF INVENTION

This invention relates to unsolvated and host-guest solvated crystalline forms of (2E,4S)-4-[(N-{[(2R)-1-isopropylpiperidin-2-yl]-carbonyl}-3-methyl-L-valyl)(methyl)amino]-2,5-dimethylhex-2-enoic acid, E7974. E7974 possesses therapeutic efficacy for the treatment of various cancers, inflammatory disorders, autoimmune disorders, and proliferative disorders as well as for the treatment and prevention of restenosis in blood vessels.

### BACKGROUND OF INVENTION

Hemiasterlin (1) was first isolated from the sponge Hemiasterella minor (class, Demospongiae; order, Hadromedidia; family, Hemiasterellidae) collected in Sodwana Bay, South Africa (see, Kashman et al. U.S. Pat. No. 5,661,175). Hemiasterlin exhibits antitumor activity against several cell lines, including human lung carcinoma, human colon carcinoma and human melanoma.

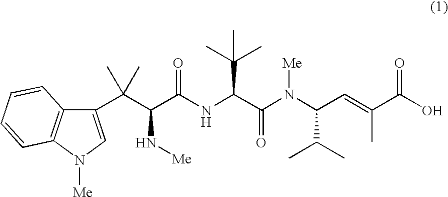

After the initial isolation and reporting of this compound, additional hemiasterlins were isolated, and several hemiasterlin derivatives were synthesized and their biological activity was also investigated. It was subsequently reported that Hemiasterlin and certain analogs thereof exhibit antimitotic activity and thus are useful for the treatment of certain cancers (see, U.S. Pat. No. 6,153,590 and PCT application WO 99/32509).

U.S. published patent application, U.S. 20040229819 A1, (which is incorporated herein by reference) discloses a number of hemiasterlin analogs and their uses. One such analog, (2E,4S)-4-[(N-{[(2R)-1-isopropylpiperidin-2-yl]-carbonyl}-3-methyl-L-valyl)(methyl)amino]-2,5-dimethylhex-2-enoic acid, E7974, possesses therapeutic activity in the treatment of various cancers, lymphoma, leukemia and multiple myeloma as well as in the treatment and prevention of restenosis of blood vessels. The synthesis of E7974 is described in Example 14 of U.S. 20040229819-A1, which identifies the compound as E807974. Example 14 reports the preparation of ER-807974 as thick oil free-base compound, not as crystalline E7974.

Although therapeutic efficacy is the primary concern for a therapeutic agent, like E7974, the salt and crystal form of a drug candidate can be critical to its development. Each salt or each crystalline form (polymorph) of a drug candidate can have different solid state (physical and chemical) properties, for example, solubility, stability, or the ability to be reproduced. These properties can impact the selection of a compound as an active pharmaceutical ingredient (API), the ultimate pharmaceutical dosage form, the optimization of manufacturing processes, and absorption in the body. Moreover, finding the most adequate form for further drug development can reduce the time and the cost of that development.

Obtaining pure crystalline forms is extremely useful in drug development. It permits better characterization of the drug candidate's chemical and physical properties. Crystalline forms often have better chemical and physical properties than the amorphous state. The crystalline form may possess more favorable pharmacology than the amorphous form or be easier to process. It may also have better storage stability.

One such physical property, which can affect processability, is the flowability of the solid, before and after milling. Flowability affects the ease with which the material is handled during processing into a pharmaceutical composition. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate. Another important solid state property of a pharmaceutical compound is its dissolution rate in aqueous fluid. The rate of dissolution of an active ingredient in a patient's stomach fluid may have therapeutic consequences since it impacts the rate at which an orally-administered active ingredient may reach the patient's bloodstream.

These practical physical properties are influenced by the solid state form of a compound, e.g., the conformation and orientation of molecules in the unit cell of the crystalline compound, or whether or not a molecule associates with solvent molecules to form a solvate. The ability of a molecule to adopt a different conformation and/or arrangement of molecules in the crystal lattice is called polymorphism. The crystalline (or polymorphic) form or solvate often has thermal behavior different from the amorphous material, another polymorphic form, or a solvate. Thermal behavior is measured in the laboratory by such techniques as capillary melting point, thermogravimetric analysis (TGA) and differential scanning calorimetry (DSC) and may be used to distinguish some polymorphic forms from others. A crystalline form or a particular polymorphic form generally possesses distinct crystallographic and spectroscopic properties detectable by powder X-ray diffraction (PXRD), single crystal X-ray crystallography, solid state NMR spectroscopy, e.g. ¹³C CP/MAS NMR, infrared spectrometry among other techniques.

### SUMMARY OF INVENTION

The invention relates to crystalline forms of (2E,4S)-4-[(N-{[(2R)-1-isopropylpiperidin-2-yl]-carbonyl}-3-methyl-L-valyl)(methyl)amino]-2,5-dimethylhex-2-enoic acid, E7974. E7974 has two unsolvated crystalline forms, M₁ and O₁. These crystalline forms, along with another form, M₂, can also form crystalline host-guest solvates where the solvent is present in cavities channels, or other void spaces within the crystal lattice. As used here, the terms cavity and/or void space also refers to channels.

The invention also relates to the therapeutic uses of the crystalline forms of E7974. Accordingly, a pharmaceutical composition containing a crystalline form of E7974 and a pharmaceutically acceptable carrier represents one embodiment of the invention. The invention further relates to methods for treating a cancer, an inflammatory disorder, an autoimmune disorder, or a proliferative disorder comprising the step of administering to a patient in need thereof a therapeutically effective amount of a crystalline form of E7974. The crystalline forms of E7974 may be administered by itself or as a pharmaceutical composition of the invention.

### BRIEF DESCRIPTION OF THE FIGURES

FIG. 1 shows the vapor sorption isotherm of crystalline E7974-form M₁_{_{—}}unsolvated from Example 2.

FIG. 2 depicts the vapor sorption isotherm of crystalline E7974-form M₁_{_{—}}unsolvated at 25° C. as a function of relative humidity (% RH) from 5% RH to 70% RH from Example 2.

FIG. 3 depicts the powder X-ray diffraction (PXRD) pattern of crystalline E7974-form M₁_{_{—}}unsolvated from multiple lots from Example 3.

FIG. 4 depicts the PXRD pattern of crystalline E7974-form M₁_{_{—}}unsolvated from Example 3.

FIG. 5 depicts the infrared spectrum of crystalline E7974-form M₁_{_{—}}unsolvated.

FIG. 6 depicts the differential scanning calorimetry (DSC) thermogram for crystalline E7974-form M₁_{_{—}}unsolvated from Example 4.

FIG. 7 depicts the ¹³C CP/MAS NMR of crystalline E7974-form M₁_{_{—}}unsolvated.

FIG. 8: depicts a schematic of the temperature profile for high throughput crystallization of E7974.

FIG. 9 depicts the PXRD pattern of crystalline E7974-form M₁_{_{—}}acetone (Plate 5: initial conc. 10% w/v).

FIG. 10 depicts the PXRD pattern of crystalline E7974-form M₁_{_{—}}1,4-dioxane (Plate 11, initial conc. 5% w/v).

FIG. 11 depicts the digital image of crystalline E7974-form M₂_{_{—}}1,4-dioxane (Plate 11, initial conc. 5% w/v).

FIG. 12 depicts the PXRD pattern of crystalline E7974-form M₂_{_{—}}1,4-dioxane (Plate 11, initial conc. 10% w/v).

FIG. 13 depicts the digital image of crystalline E7974-form M₂_{_{—}}1,4-dioxane (Plate 11, initial conc. 10% w/v).

FIG. 14 depicts the PXRD pattern of crystalline E7974-form M₂_{_{—}}THF (Plate 1, initial conc. 10% w/v).

FIG. 15 depicts the PXRD pattern of crystalline E7974-form M₂_{_{—}}Acetone (Plate 11, initial conc. 10% w/v).

FIG. 16 depicts the digital image of crystalline E7974-form M₂_{_{—}}acetone (Plate 11, initial conc. 10% w/v).

FIG. 17 depicts the PXRD pattern of crystalline E7974-form M₂_{_{—}}acetone (Plate 12, initial conc. 5% w/v).

FIG. 18 depicts the digital image of crystalline E7974-form M₂_{_{—}}acetone (Plate 12, initial conc. 5% w/v).

FIG. 19 depicts the PXRD pattern of crystalline E7974-form M₂_{_{—}}amyl ether (Plate 2, initial conc. 5% w/v).

FIG. 20 depicts the PXRD pattern of crystalline E7974-form M₂_{_{—}}nitromethane (Plate 5, initial conc. 5% w/v).

FIG. 21 depicts the PXRD pattern of crystalline E7974-form M₂_{_{—}}ethyl acetate/n-heptane (50:50) (Plate 7, initial conc. 5% w/v).

FIG. 22 depicts the digital image of crystalline E7974-form M₂_{_{—}}ethyl acetate/n-heptane (50:50) (Plate 7, initial conc. 5% w/v).

FIG. 23 depicts the PXRD pattern of crystalline E7974-form M₂_{_{—}}ethyl acetate/n-heptane (50:50) (Plate 7, initial conc. 10% w/v).

FIG. 24 depicts the digital image of crystalline E7974-form M₂_{_{—}}ethyl acetate/n-heptane (50:50) (Plate 7, initial conc. 10% w/v).

FIG. 25 depicts the PXRD pattern of crystalline E7974-form O₁_{_{—}}toluene (Plate 8, initial conc. 5% w/v).

FIG. 26 depicts the digital image of crystalline E7974-form O₁_{_{—}}toluene (Plate 8, initial conc. 5% w/v).

FIG. 27 depicts the PXRD pattern of crystalline E7974-form O₁_{_{—}}toluene (Plate 8, initial conc. 10% w/v)

FIG. 28 depicts the digital image of crystalline E7974-form O₁_{_{—}}toluene (Plate 8, initial conc. 10% w/v).

FIG. 29 depicts the PXRD pattern of crystalline E7974-form O1_{_{—}}nitrobenzene (Plate 4, initial conc. 10% w/v).

FIG. 30 depicts the digital image of crystalline E7974-form O₁_{_{—}}nitrobenzene (Plate 4, initial conc. 10% w/v).

FIG. 31 depicts the PXRD pattern of crystalline E7974-form O₁_{_{—}}nitrobenzene (Plate 9, initial conc. 10% w/v).

FIG. 32 depicts the Digital image of crystalline E7974-form O₁_{_{—}}nitrobenzene (Plate 9, initial conc. 10% w/v).

FIG. 33 depicts the PXRD pattern of crystalline E7974-form O₁_{_{—}}trifluroemethyl toluene (Plate 6, initial conc. 10% w/v).

FIG. 34 depicts the PXRD pattern of crystalline E7974-form O₁_{_{—}}water/ethanaol (10:90) (Plate 12, initial conc. 10% w/v).

FIG. 35 depicts the experimental PXRD pattern of crystalline E7974-form M₂_{_{—}}amyl ether (top, Plate 2, low concentration) and the calculated PXRD patterns based on the determined structures of crystalline E7974-form M₂_{_{—}}amyl ether and of crystalline E7974-form M₂_{_{—}}amyl ether considering preferred orientation effects involving the (020) crystallographic plane (bottom pattern).

FIG. 36 depicts the crystal packing of form J viewed down c-axis. Amyl ether molecules are incorporated in the structure cavities.

FIG. 37 depicts the crystal packing of crystalline E7974-form O₁_{_{—}}nitrobenzene with nitrobenzene molecules incorporated in the structure cavities.

FIG. 38 depicts PXRD patterns of crystalline E7974-form O₁_{_{—}}nitrobenzene (from top: Plate 2, high concentration, Plate 9 high concentration) and of crystalline E7974-form O₁_{_{—}}nitrobenzene (Plate 011 high concentration). The bottom pattern is the calculated pattern based on the crystal structure of crystalline E7974-form O₁_{_{—}}nitrobenzene. The arrows indicate the additional peaks present in the patterns.

FIG. 39 depicts calculated PXRD patterns from the respectively determined crystal structures (from top to bottom): of crystalline E7974-form O₁_{_{—}}TBME, (Plate 8, low concentration), crystalline E7974-form O₁_{_{—}}nitrobenzene, (Plate 9, low concentration, crystallization T=25° C.) and crystalline E7974-form O₁_{_{—}}nitrobenzene, (Plate 3, high concentration, crystallization T=5° C.).

FIG. 40 depicts the IR spectrum of crystalline E7974-form M₁_{_{—}}acetonitrile from a sealed, spinning capillary tube.

FIG. 41 depicts the PXRD pattern of crystalline E7974-form M₁_{_{—}}acetonitrile from a sealed, spinning capillary tube.

FIG. 42 shows the PXRD pattern of crystalline E7974-form M₂_{_{—}}1,4 dioxane.

FIG. 43 shows the infrared spectrum of crystalline E7974-form M₂_{_{—}}1,4 dioxane.

FIG. 44 shows the DSC thermogram of crystalline E7974-form M₂_{_{—}}1,4-dioxane.

FIG. 45 shows the PXRD pattern of crystalline E7974-form O₁_{_{—}}unsolvated.

FIG. 46 shows the infrared spectrum of crystalline E7974-form O₁_{_{—}}unsolvated.

FIG. 47 depicts the 13C CP/MAS NMR of crystalline E7974-form O₁_{_{—}}unsolvated.

FIG. 48 shows the DSC thermogram of crystalline E7974-form O₁_{_{—}}unsolvated.

FIG. 49 shows the PXRD pattern of crystalline E7974-form O₁_{_{—}}toluene.

FIG. 50 shows the infrared spectrum of crystalline E7974-form O₁_{_{—}}toluene.

FIG. 51 shows the DSC thermogram of crystalline E7974-form O₁_{_{—}}toluene.

### DETAILED DESCRIPTION OF THE INVENTION

(2E,4S)-4-[(N-{[(2R)-1-isopropylpiperidin-2-yl]carbonyl}-3-methyl-L-valyl)(methyl)amino]-2,5-dimethylhex-2-enoic acid (IUPAC nomenclature), E7974, has the following chemical formula (2). 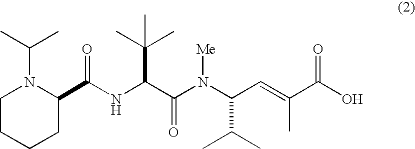
The CAS chemical name for E7974 is 2-Hexenoic acid, 4-[[(2S)-3,3-dimethyl-2-[[[(2R)-1-(1-methylethyl)-2-piperidinyl]carbonyl]amino]-1-oxobutyl]methylamino]-2,5-dimethyl 2E,4S). Its CAS Registry Number is 610787-07-0. E7974 is the zwitterionic form of the compound.

E7974 is useful as a therapeutic agent for the treatment of various cancers, inflammatory disorders, autoimmune disorders, and proliferative disorders. More specifically, E7974 can be used for the treatment of diseases and disorders including, but not limited to prostate, breast, colon, bladder, cervical, skin, testicular, kidney, ovarian, stomach, brain, liver, pancreatic and esophageal cancer, lymphoma, leukemia and multiple myeloma. The chemical synthesis and anti-tumor activity of E7974 were the subject of three posters presented at the 96th Annual Meeting of the American Association for Cancer Research (AACR), Apr. 16-20, 2005, Anaheim, Calif.: 1) Tubulin-based Antimitotic Mechanism of Novel Hemiasterlin Analog E7974, G. Kuznetsov et al., Abstract No. 3436; 2) Synthetic Analogs of the Natural Marine Product Hemiasterlin: Optimization and Discovery of E7974, a Novel and Potent Anti-tumor Agent, J. Kowalczyk et al., Abstract No. 1212; and 3) In vitro and in vivo antitumor activities of novel hemiasterlin analog E7974, G. Kuznetsov et al., Abstract No. 3432. E7974 can also be used to treat and prevent restenosis of blood vessels subject to traumas such as angioplasty and stenting.

### 1. CRYSTALLINE FORMS OF E7974

This invention relates to crystalline forms of E7974, unsolvated crystalline forms and host-guest solvates of those crystalline forms. Unless a specific form designation is given, the term “crystalline E7974” refers to all crystalline forms of E7974 described here. There are two monoclinic crystalline forms, M₁ and M₂ and one orthorhombic crystalline form, O₁. The M₁ and O₁ crystalline forms exist as unsolvated crystalline forms. Each of these is described below. The space group designations, monoclinic and orthorhombic, generally refer to the host crystal space group. Upon solution, the specific group of the host-guest soluate may change somewhat and be substantially the same as that of the host.

The M₁, M₂ and O₁ crystalline forms have the ability to incorporate solvent molecules into their crystal lattices without losing crystallinity. These solvates are “host-guest” in that the solvent is incorporated into a cavity, (also called a void space or channel) in the crystalline E7974 lattice.

### 2. CRYSTALLINE E7974-FORM M₁_{_{—}}UNSOLVATED

Crystalline E7974-form M₁ is prepared by crystallizing crude E7974 in acetonitrile with heating up to reflux and then slowly cooling to allow crystal formation. In a preferred method, crude E7974 may be first crystallized from acetonitrile at room temperature, preferably 25° C., and then recrystallizing in acetonitrile with heating to reflux and slow cooling. Drying solvated forms of crystalline form M₁ also yields the unsolvated crystalline form M₁.

Crystalline E7974-form M₁ possesses superior processability, purification controls (by recrystallization), and solid-state stability. As described below in the Examples and shown in the Figures, crystalline E7974-form M₁ was characterized by X-ray powder diffraction (XRD), single crystal X-ray diffraction, infrared spectroscopy, solid state ¹³C NMR, thermal analyses and hygroscopicity measurements.

### 3. CRYSTALLINE E7974-FORM O₁_{_{—}}UNSOLVATED

Crystalline E 7974-form O₁ is a second unsolvated crystal form of E7974. As discussed below, form O₁ is prepared by dissolving E7974 in various solvents and then drying the resulting crystalline solid to remove the solvent and yield the unsolvated form O₁. The Examples and Figures below characterize form O₁ using X-ray powder diffraction (XRD), single crystal X-ray diffraction, infrared spectroscopy, solid state ¹³C NMR spectroscopy, thermal analyses and hygroscopicity measurements. The approximate size of the cavity was calculated using a virtual solvent-free structure (crystalline E7974-O₁-nitrobenzene by excluding the nitrobenzene molecules from the crystal structure and keeping the unit cell parameters unmodified). The Volume of the Total Potential Solvent Area is 936.2 Å³ versus a unit cell volume of 3260.3 Å³, which means that 28.7% of the unit cell volume of O₁ form simulated solvent-free structure should be accessible for solvent molecules.

### 4. HOST-GUEST SOLVATES OF CRYSTALLINE E7974-FORMS M₁_{_{—}}SOLVENT, M₂_{_{—}}SOLVENT, AND O₁_{_{—}}SOLVENT

Crystalline forms of E7974 of the invention contain cavities, channels or void spaces (all of which are referred to here as cavities), in the crystal structure and form solvated crystalline forms as “host-guest solvates” in which solvent molecules are present within the cavities. These crystalline forms of E7974 form host-guest solvates with organic solvents. The solvent may be present in a stoichiometric amount or a non-stoichiometric amount. A “non-stoichiometric solvate” is one where different preparation methods or processing of the material result in a non-discrete (or continuous) change in the solvent stoichiometry relative to the E7974 molecules in the crystal. Some crystalline forms of the invention have cavities which may contain organic solvent molecules. Both forms M₁ and O₁ form host-guest solvates. In addition, another monoclinic crystalline form, M₂, exists as a host-guest solvated form.

There is no particular limitation on the organic solvent which may be solvated within the cavity of the crystalline E7974, other than that the host-guest solvate be a crystalline solid. The organic solvent may be a single solvent, a mixture of organic solvents, or an aqueous mixture containing the organic solvent(s). The solvent is typically the solvent used to manufacture crystalline E7974 or a pharmaceutical composition containing E7974. Accordingly the organic solvent forming the host-guest solvate is often one used in the synthesis or purification of E7974, which may be advantageous for the process. Drying the host-guest solvate yields the unsolvated form or, in the case of solvated form M₂, the unsolvated form M₁. The crystalline host-guest solvates of the invention may exist as mixtures of forms, including mixtures of solvated and unsolvated forms.

Suitable solvents used to form host-guest solvates include, but are not limited to, 1,4-dioxane; 1-bromopropane; 1-bitropropane; 2-butoxyethyl acetate; acetone, acetonitrile; amyl ether; chlorobenzene; chloroform, cyclohexanone; dichloromethane (DCM); diisobutyl ketone; diisopropylether; N₁N-dimethylacetamid (DMA); dimethylformamide (DMF); ethylacetate/n-heptane (50:50); ethylacetate; isophorone; methyl isobutyl ketone (MIBK); n-butylacetate; nitrobenzene; nitromethane; t-butyl methylether (TBME); 2,2,2-trifluroethenol

A pharmaceutical composition of the invention may be any pharmaceutical form which contains one of the crystalline forms of E7974. The pharmaceutical composition may be a solid form, a liquid suspension, an injectable composition, a topical form, or a transdermal form. These pharmaceutical forms are disclosed in U.S. 20040229819 A1, which is incorporated here by reference.

Depending on the type of pharmaceutical composition, the pharmaceutically acceptable carrier may be chosen from any one or a combination of carriers known in the art. The choice of the pharmaceutically acceptable carrier depends upon the pharmaceutical form and the desired method of administration to be used. For a solid pharmaceutical composition of the invention, that is one having a crystalline form of E7974, a carrier should be chosen that maintains the particular crystalline form of E7974 used. In other words, for solid pharmaceutical compositions the carrier should not substantially alter the crystalline form of E7974. Nor should the carrier be incompatible with E7974, such as by producing any undesirable biological effect or otherwise interacting in a deleterious manner with any other component(s) of the pharmaceutical composition.

The pharmaceutical compositions of the invention are preferably formulated in unit dosage form for ease of administration and uniformity of dosage. A “unit dosage form” refers to a physically discrete unit of therapeutic agent appropriate for the patient to be treated. It will be understood, however, that the total daily dosage of E7974 and its pharmaceutical compositions according to the invention will be decided by the attending physician within the scope of sound medical judgment.

Because the crystalline forms of E7974 are more easily maintained during their preparation, solid dosage forms are a preferred form for the pharmaceutical composition of the invention. Solid dosage forms for oral administration, such as capsules, tablets, pills, powders, and granules, are particularly preferred. In such solid dosage forms, the active compound is mixed with at least one inert, pharmaceutically acceptable carrier such as sodium citrate or dicalcium phosphate. The solid dosage form may also include one or more of: a) fillers or extenders such as starches, lactose, sucrose, glucose, mannitol, and silicic acid; b) binders such as, for example, carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) dissolution retarding agents such as paraffin; f) absorption accelerators such as quaternary ammonium compounds; g) wetting agents such as, for (TFE); tetrahydrofuron (THF); toluene; trichloroethylene; trifluomethane toluene; water/2-propanol (10:90); water/2-propanol (20:80); water/acetone (10:90); water/acetone (20:80); water/acetonitrile (10:90); water/ethanol (10:90); and water/ethanol (20:80). It is generally preferred that the organic solvent is a pharmaceutically acceptable solvent. Preferred organic solvents for host-guest solvates of crystalline E7974-form M₁ are the acetone and acetonitrile solvates. For host-guest solvate of crystalline E7974-form M₂, the following solvents are preferred: 1,4-dioxane, ethylacetate/n-heptane (50:50), acetone, and nitromethane. The preferred solvents for the host-guest solvates of crystalline E7974-form O₁ are toluene, water/ethanol (10:90), TBME, and nitrobenzene.

### 5. PHARMACEUTICAL COMPOSITIONS

The invention relates to pharmaceutical compositions comprising a therapeutically effective amount of a crystalline form of E7974 and a pharmaceutically acceptable carrier. As discussed above, E7974 possesses biological properties making it useful for the treatment of cancer, inflammatory, autoimmune, and/or proliferative diseases and disorders as well as the treatment and prevention of restenosis in blood vessels. Pharmaceutical compositions for the treatment of those diseases and disorders contain a therapeutically effective amount of a crystalline form of E7974 as appropriate for treatment of a patient with the particular disease or disorder.

A “therapeutically effective amount” of E7974 in a crystalline form of the invention (discussed here concerning the pharmaceutical compositions and below concerning the methods of treatment according to the invention) refers to an amount sufficient to reduce the effects of an inflammatory or autoimmune response or disorder; an amount sufficient to prevent, kill, or inhibit the growth or speed of tumor cells; or an amount sufficient to treat or prevent restenosis of blood vessels. The actual amount required for treatment of any particular patient will depend upon a variety of factors including the disorder being treated and its severity; the specific pharmaceutical composition employed; the age, body weight, general health, sex and diet of the patient; the mode of administration; the time of administration; the route of administration; and the rate of excretion of E7974; the duration of the treatment; any drugs used in combination or coincidental with the specific compound employed; and other such factors well known in the medical arts. These factors are discussed in Goodman and Gilman's “The Pharmacological Basis of Therapeutics”, Tenth Edition, A. Gilman, J. Hardman and L. Limbird, eds., McGraw-Hill Press, 155-173, 2001, which is incorporated herein by reference.

example, cetyl alcohol and glycerol monostearate; h) absorbents such as kaolin and bentonite clay; and i) lubricants such as talc, calcium stearate, magnesium stearate, solid polyethylene glycols, sodium lauryl sulfate. The solid dosage forms may also comprise buffering agents. They may optionally contain opacifying agents and can also be of a composition that they release the active ingredient(s) only, or preferentially, in a certain part of the intestinal tract, optionally, in a delayed manner Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Solid dosage forms of pharmaceutical compositions of the invention can also be prepared with coatings and shells such as enteric coatings and other coatings well known in the pharmaceutical formulating art.

Crystalline E7974 can be in a solid micro-encapsulated form with one or more carriers as discussed above. Microencapsulated forms of crystalline forms of E7974 may also be used in soft and hard-filled gelatin capsules with excipients such as lactose or milk sugar as well as high molecular weight polyethylene glycols and the like.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension, by the use of a crystalline form, or by the use of an amorphous material with poor water solubility. The rate of absorption of the drug then depends upon its rate of dissolution that, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include (poly(orthoesters) and poly(anhydrides)). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release E7974.

Crystalline forms of E7974 according to the invention may also be used to formulate or be formulated in an autoclavable liquid formulation. Exemplary aqueous development formulations (1 mg/ml E7974) include 1) isotonic 5% dextrose, 20 mM citrate buffer, pH 4.5; 2) non-isotonic, 20 mM citrate buffer, pH 4.5; and 3) 0.9% NaCl, 20 mM phosphate buffer, pH 7. All three autoclaved formulations show good storage stability.

### 6. METHODS OF TREATMENT USING THE CRYSTALLINE FORMS OF E7974

The invention also provides methods for and the use of crystalline E7974 in the treatment of proliferative disorders, inflammatory or autoimmune disorders, as well as to treat or prevent restenosis of blood vessels. Proliferative disorders include cancers, such as colorectal cancer, glioblastoma multiforme (GBM), breast, prostate, non-small cell lung cancer, esophageal/gastic cancer and hepatocellular cancers or tumors. Some tumors may be resistant to certain drugs, such as multi-drug resistant, or taxane-resistant tumors. Crystalline E7974 and pharmaceutical compositions containing it may, according to the invention, be administered using any amount, any form of pharmaceutical composition and any route of administration effective for the treatment. After formulation with an appropriate pharmaceutically acceptable carrier in a desired dosage, the pharmaceutical compositions of this invention can be administered to humans and other animals orally, rectally, parenterally, intraveneously, intracistemally, intravaginally, intraperitoneally, topically (as by powders, ointments, or drops), bucally, as an oral or nasal spray, or the like, depending on the location and severity of the condition being treated. In certain embodiments, crystalline forms of E7974 according to the invention may be administered at dosage levels of about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject. Crystalline forms of the invention may be administered alone or in combination with other active agents such as anti-cancer agents including anthracyclines, gemcitabine, cisplatin, carboplatin, doctaxel, or a combination of active agents. The combination may be in the form of a composition of the invention comprising one, two or more additional active agents. Alternatively, the additional active agents may be administered separately, before, during or after administration of a composition of the invention. Thus, the various crystalline forms of the invention may be used in the manufacture of a medicament for the treatment of proliferative disorder including cancer, an inflammatory or autoimmune disorder, or restenosis.

### 7. EXAMPLES

### Example 1

### Preparation of Crystalline E7974-form M₁_{_{—}}unsolvated

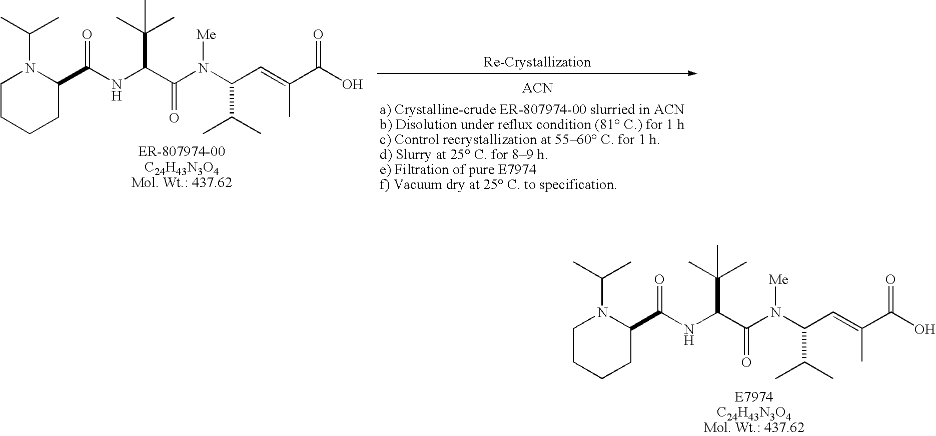

To prepare crystalline E7974-form M₁_{_{—}}unsolvated, crude ER-807974-00, a zwitterion, was dissolved in acetonitrile (ACN) under reflux condition at 81° C. and held at such temperature for a period of 0.5 to 1 hour. The re-crystallization was controlled by slow solution cooling to 65° to 55° C. The mixture was stirred at that temperature range for 1 hour. Finally, the slurry is stirred at 20° C. for 8 hours and the E7974 was harvested by filtration. The filter cake was washed with cold acetonitrile and dried under vacuum at 25° C. until dry. These crystallization conditions consistently gave a crystalline solid form with reproducible powder X-ray diffraction (PXRD) patterns. See Example 3, below.

### Example 2

### Hygroscopicity Studies

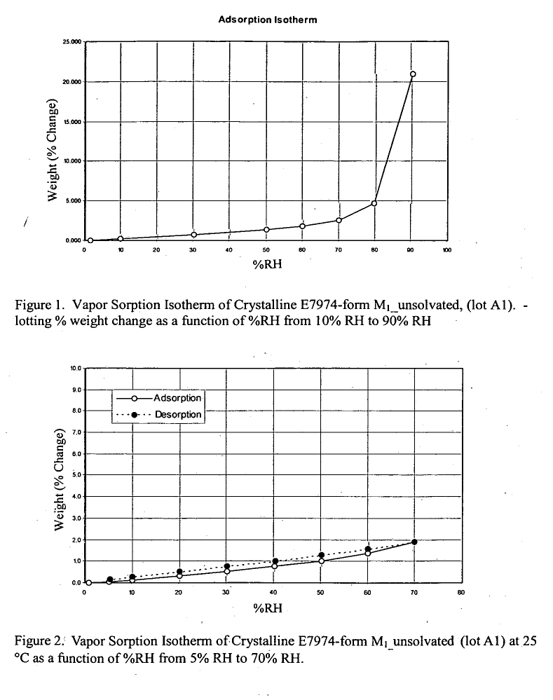

Crystalline E7974-from M₁_{_{—}}unsolvated was found to be a slightly hygroscopic compound that deliquesces at high relative humidity (% RH) (see FIG. 1). In order to avoid deliquescence and to observe the desorption of water, a separate experiment which investigated hygroscopicity up to 70% RH was performed (see FIG. 2). A 1.9% increase in weight was observed at 70% RH verifying that the compound is non-hygroscopic. According to the established criteria (Tsunakawa et al., IYAKUHIN KENKYU, 22 (1), 173-176 (1991)), it is defined that a hygroscopic material shows not less than 3.0% increase in the water content after storage at 75% relative humidity for a week. The water adsorption was reversible up to 70% RH. No plateaus were observed in the desorption curve therefore.

### Example 3

### Characterization of Unsolvated Crystalline E7974-form M1 by Powder X-ray Diffraction (PXRD) and Infrared (IR) Spectroscopy

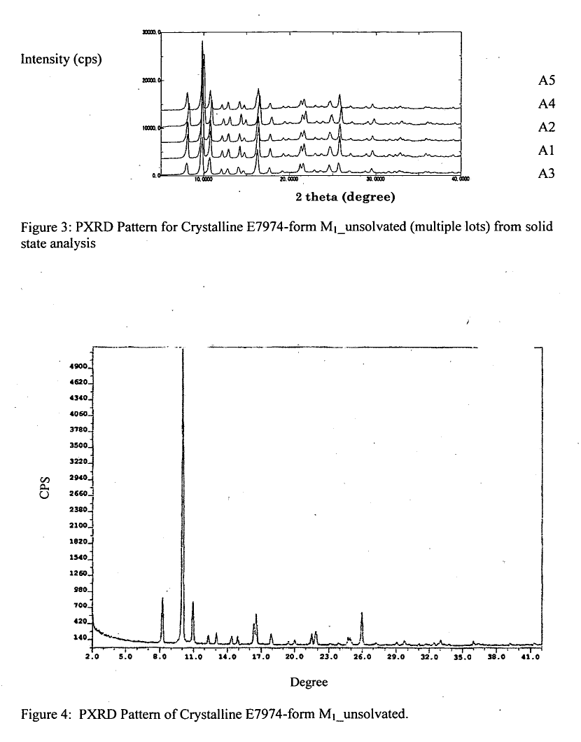

Crystalline E7974-form M₁ was characterized by powder X-ray diffraction (PXRD). Crystalline E7974-form M₁ powder was placed on the sample platform of an X-ray powder diffractometer (RINT-2000, Rigaku, Japan) and analyzed under the conditions shown in Table 1. FIG. 3 shows the PXRD pattern for five lots (A1-A5) of crystalline E7974-form M₁. All five lots showed consistent PXRD patterns.

TABLE 1
Powder X-ray Diffraction Measurement Conditions

Target: Cu
Detector: Scintillation counter
Tube voltage: 40 kV
Tube current: 200 mA
Slit: DS 1/2°, RS 0.3 mm, SS 1/2°
Scan speed: 2°/min
Step/Sampling: 0.02°
Scan range: 5 to 40°
Sample holder: Glass holder (diameter: 5 mm)
Goniometer: Vertical goniometer
Monochromater: used

FIG. 4 also shows the PXRD of unsolvated crystalline E7974-form M₁. Powder X-ray diffraction (PXRD) data were collected at ambient temperature on a Scintag X₂ θ/θ diffractometer (40000065), operating with copper radiation at 45 kV and 40 mA, using a Thermo ARL Peltier-cooled solid-state detector. Source slits of 2 and 4 mm, and detector slits of 0.5 and 0.3 mm were used for data collection. The PXRD unit is equipped with a Scintag 6 position sample changer (autosampler), PC with Windows NT 4.0 operating system, and DMSNT software version 1.36b. The PXRD unit was aligned upon installation using National Bureau of Standards (now NIST) silicon powder as a standard. The result of the alignment was then logged in the PXRD calibration logbook. The alignment of the PXRD unit is rechecked annually and under any of the following conditions: (1) A new sample stage is installed; (2) The Scintag X₂ is moved. Table 2 lists additional parameters used to collect the PXRD data.

TABLE 2
Powder X-ray Diffraction Measurement Conditions

Scan speed: 1°/min
Step/Sampling: 0.02°
Scan range: 2 to 42°
Sample holder: Stainless Steel Holder (diameter: 5 mm)
Goniometer: Vertical goniometer

Table 3 identifies the peaks in the PXRD pattern in FIG. 4. Table 4 is a listing of preferred characteristic peaks of crystalline E7974-form M₁_{_{—}}unsolvated. In a further preferred embodiment, the unsolvated form M₁ is characterized as having at least four peaks in its powder X-ray diffraction pattern selected from the group consisting of the following 2Θ values: 8.2±0.2, 10.0±0.2, 10.9±0.2, 13.0±0.2, 14.3±0.2, 16.3±0.2, and 17.9±0.2. Any four or more of which should sufficiently identify crystalline E7974 form M₁_{_{—}}unsolvated.

TABLE 3

| Peak Position | Relative |
|---|---|
| Deg. 2Θ ± 0.2 | Intensity. |
| 8.2 | 536.13 |
| 10.0 | 3532.27 |
| 10.4 | 24.78 |
| 10.9 | 493.75 |
| 12.3 | 103.03 |
| 13.0 | 135.77 |
| 14.3 | 91.68 |
| 14.9 | 103.08 |
| 16.3 | 264.43 |
| 16.5 | 389.38 |
| 17.9 | 123.57 |
| 19.4 | 42.1 |
| 20.0 | 59.22 |
| 21.5 | 133.68 |
| 21.8 | 149.97 |
| 24.7 | 102.57 |
| 24.9 | 104.37 |
| 25.9 | 448.65 |
| 29.0 | 32.02 |
| 29.7 | 59.98 |
| 32.4 | 26.93 |
| 33.0 | 64.1 |
| 35.9 | 64.85 |

TABLE 4
Characteristic PXRD Peaks of
Unsolvated Crystalline E7974-form
M₁
2 Θ (degree)
8.2 ± 0.2
10.0 ± 0.2
10.9 ± 0.2
12.2 ± 0.2
13.0 ± 0.2
14.3 ± 0.2
14.9 ± 0.2
16.3 ± 0.2
16.5 ± 0.2
17.9 ± 0.2

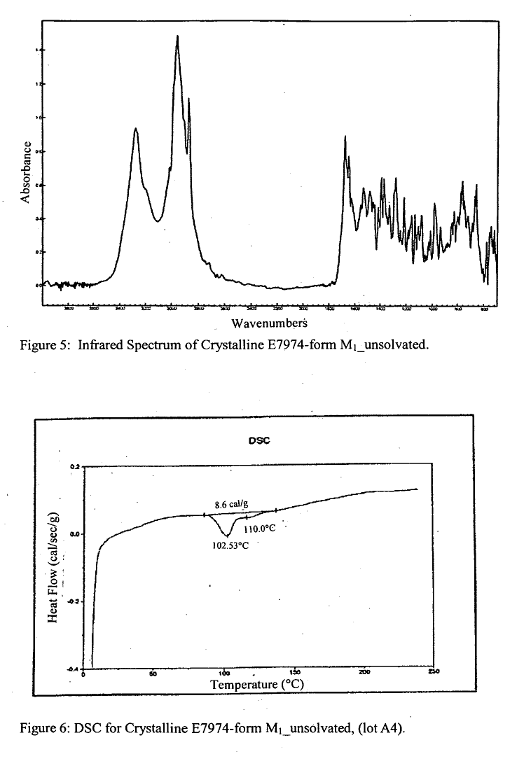

FIG. 5 depicts the infrared spectrum of crystalline E7974-form M₁_{_{—}}unsolvated. The spectrum was run on a Bio Rad FTS-6000 FTIR instrument. The spectrum was collected using Diffused Reflectance. A background was collected using Poatassium Bromide at 64 co-scans and a resolution of 2 cm⁻¹. The spectrum was collected at 16 co-scans and a resolution of 2 cm¹.

### Example 4

### Characterization by Differential Scanning Calorimetry (DSC)

Solid-state characterization of crystalline E7974-form M₁_{_{—}}unsolvated was determined by Differential Scanning Calorimetry (DSC, capillary technique). Table 5 lists the conditions used. FIG. 6 shows the thermograms of unsolvated crystalline E7974-form M₁ with a broad endothermic peak at 102.53° C., giving a melting point of 102.5° C. The analyzed sample of E7974 melted with overlapping events at 110° C. (onset temp.) absorbing an approximate total of +8.6 cal/g in the presence of nitrogen. DSC data of this sample was collected at different heating rates to verify that the overlapping peak was not due to a metastable form. The overlapping peak was not observed when a fast heating (25° C./min) was used.

TABLE 5

| | |
|---|---|
| Sample: | E7974-form M₁_unsolvated |
| Sample Size: | 3.26000 mg |
| Instrument Type: | 2920 DSC V2.5F |
| Pan Type: | Aluminum |
| Gas 1: | Nitrogen 50 |
| Method: | Aluminum Pan 210° C. @ 10° C./min. |

### Example 5

### ¹³C CP/MAS NMR Spectra of Crystalline E7974-Form M₁_{_{—}}unsolvated

¹³C CP/MAS NMR spectra were acquired at 100.6 MHz for ¹³C using a Varian NMR spectrometer equipped with a Varian 7 mm CPMAS probe. All equipment was thoroughly cleaned before packing and unpacking each sample. A sample of crystalline E7974-form M₁_{_{—}}unsolvated was packed in a zirconia rotor. No excessive force (e.g. grinding) was used to pack the sample in the rotor in order to minimize potential polymorphic conversion. The sample was spun at the magic angle at 5.0 kHz. Spectra were acquired with total sideband suppression (TOSS), a 4 s recycle delay, and a decoupling field of approximately 60 kHz. The ¹H 90° pulse was ˜4 μs, and the contact time was 3 ms. Spectra were externally referenced to tetramethylsilane using the methyl peak of hexamethylbenzene (17.35 ppm).

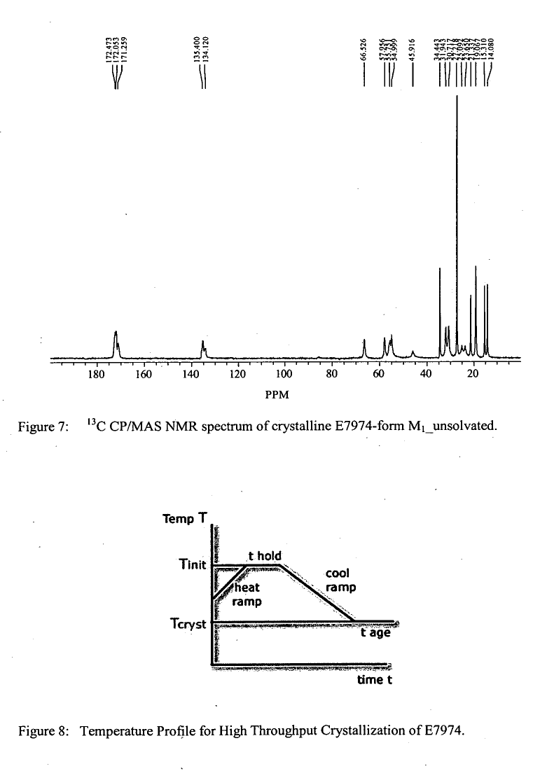

FIG. 7 shows the resulting ¹³C CP/MAS NMR spectrum of crystalline E7974-form M₁_{_{—}}unsolvated, with peak positions indicated on the spectrum. Preferred characteristic peaks for the identification of crystalline E7974-form M₁_{_{—}}unsolvated can be found in the region of approximately 14-35 ppm. Particularly preferred characteristic peaks for crystalline E7974-form M₁_{_{—}}unsolvated appear at 14.1; 15.3; 19.1, 21.3, 23.7, and 27.2 ppm any three or more of which should sufficiently identify crystalline E7974 form M₁_{_{—}}unsolvated. Chemical shifts are reported to be within ±0.3 ppm.

In general, these preferred peaks can be observed in the solid-state ¹³C NMR spectrum of an intact tablet without significant overlap from other peaks. The reason is that many common excipients, which are the ingredients added to the active pharmaceutical ingredient (API) to make the pharmaceutical tablet composition, will also show up in the solid-state ¹³C NMR spectrum. Given their chemical nature, the resonances for these excipients generally appear between 50 and 110 ppm in the ¹³C-NMR spectrum. The excipient peaks can be significantly more intense than the peaks from the API if the tablet composition is dominated by excipients. For this reason the preferred range in the solid-state ¹³C NMR spectra to identify and compare peaks from a crystalline E7974-form M₁_{_{—}}unsolvated is below 50 or above 120 ppm.

### Example 6

### Solid State Stability Studies

The solid-state stability of crystalline E7974-form M₁_{_{—}}unsolvated was evaluated for 21 days. No significant changes were observed in the impurity profile for a sample that was shielded from light and stored at 25 and 60° C. After 21 days at 25° C. and visible light, two new impurity peaks were observed at the 0.05 and 0.09% levels. In addition, a different peak appeared at only a 0.12% level for sample stored at 40° C. and exposed to 75% relative humidity. The results are shown in Table 6.

TABLE 6
Solid State Stability of Crystalline E7974-form M₁_{_{—}}unsolvated¹

| Storage condition | Time | Wt/Wt² | Total impurities³ |
|---|---|---|---|
| Initial | NA | NA | 0.72% |
| −20° C., light shield | 21 d | NA | 0.72% |
| 25° C., light shield | 21 d | 100.3% | 0.76% |
| 25° C., visible light⁴ | 21 d | 99.9% | 0.87% |
| 40° C., 75% RH | 21 d | 102.2% | 0.89% |
| 60° C., light shield | 21 d | 100.9% | 0.78% |

¹Three preparations were prepared at each condition and the averages of the results are listed here.
²Light shielded sample stored at −20° C. was used as control for weight/weight analyses.
³Sum of all impurities >0.05% as determined by HPLC area-%.
⁴Sample solutions received 6.8 million lux-hours visible light irradiation. ICH guidelines, recommend samples receive no less than 1.2 million lux-hours visible light irradiation for regulated drug product stability filings.

### Example 7

### Procedure for Re-Crystallization of E7974 to Produce Host-Guest Solvated Crystal Forms

The following procedure may be used to prepare solvated crystalline forms of E7974 M₁, M₂, and O₁. Forms M₂_{_{—}}1,4-dioxane, M₂-nitromethane and O₁_{_{—}}toluene are exemplified. Preparation of host-guest solvates of crystalline E7974 involve the following steps:
1) Starting material E7974 (preferably crystalline E7974 form M₁_{_{—}}unsolvated) is charged into the reactor (see Table 7):
2) The appropriate solvent is charged into the reactor (see Table 7).
3) The mixture is stirred at room temperature.
4) The mixture is heated at a rate of 5° C./min to or near the boiling point of the solvent used.
5) The mixture is stirred at indicated temperature (see Table 7) for 30 min. Complete dissolution should be observed at this point. The mixture may be filtered to remove any un-dissolved material. A polish hot-filtration might be required if a suspension is observed.
6) The solution is cool at a rate of 5° C./min to the crystallization temperature (see Table 7). Crystallization should be observed at this point.
7) The re-crystallized product is aged for the appropriate time at the indicated temperature (see Table 7).
8) The crystallized material is filtered through a suitable filter (fritted glass class D filter is recommended).
9) A sample of the “wet-cake” is analyzed by PXRD to confirm the crystal form (M₁_{_{—}}solvent, M₂_{_{—}}solvent or O₁_{_{—}}solvent). (See Table 8).

10) The filtered solid is partially dried under nitrogen flow for 30 to 60 min.

TABLE 7

| | | Starting | | | Heating | | Holding | Cooling | Cryst. | Aging |
|---|---|---|---|---|---|---|---|---|---|---|
| | | Material | Sol.Vol. | Conc. | Rate | Tmax | Time | Rate | Temp. | Time |
| Entry | Solvent | Wt.(mg) | (mL) | (mg/mL) | (° C./min) | (° C.) | (min) | (° C./h) | (° C.) | (h) |
| 1 | 1,4-dioxane | 500 | 5.0 | 100 | 5 | 75¹ | 30 | 5 | 25 | 1 |
| 2 | Nitromethane | 500 | 10.0 | 50 | 5 | 75¹ | 30 | 5 | 5 | 72 |
| 3 | Toluene | 500 | 10.0 | 50 | 5 | 85² | 30 | 5 | 5 | 72 |

¹Dissolution should be observed from 63° C. to 70° C.
²Sample was not dissolved after 1 h at 75° C.

Observations: Recovery yield for toluene crystallization (entry 3) was 92%. Mediocre to poor recovery was observed from 1,4-dioxane or nitromethane. Solution turned yellow when it was heated >100° C. for 15 min., possible indication of decomposition.

TABLE 8

| | Polymorphic | | Crystallinity* | Conc. | T Cryst. | Ageing Time |
|---|---|---|---|---|---|---|
| Entry | Class | Form | (PXRD) | (mg/mL) | (° C.) | (h) |
| 1 | Monoclinic M₂ | M₂_1,4-dioxane | Very good | 100 | 25 | 1 |
| 2 | Monoclinic M₂ | M₂_nitromethane | Good | 50 | 5 | 72 |
| 3 | Orthorhombic O₁ | O₁_toluene | Good | 50 | 5 | 72 |

*Qualitative crystallinity was established by visual inspection of the PXRD patterns

Unsolvated crystalline E7974 forms M₁ and O₁ may be prepared by drying the host-guest crystalline solvates. The solvated product is fully dried under high vacuum at 25° C. to constant weight. A sample of the dried product is analyzed by PXRD to confirm the crystal form (M₁_{_{—}}unsolvated or O₁_{_{—}}unsolvated). Drying crystalline E7974 M₂_{_{—}}solvent forms yields crystalline E7974 M₁_{_{—}}unsolvated.

### Example 8

### High Throughput Crystallization Studies of E7974

High throughput crystallization studies were performed using crystalline E7974-form M₁ as the starting material. The 96-well plates were divided into two parts in which each part contained a different concentration of starting material in solvent: 50 mg/ml (columns A to F) and 100 mg/ml (columns G to L) (see Table 9). A stock solution of E7974 in methanol (100 mg/ml) was used for dosing the starting material in the well plates (20 μL for the low concentration wells, 5% w/v, and 40 μL for the high concentration wells, 10% w/v). The plates containing the stock solution were placed in a vacuum chamber (1.3 kPa) at room temperature for 48 h. After the stock solvent was evaporated different solvents were added and each well was individually sealed. The 96-well plates containing E7974 and crystallization solvents were subjected to a series of temperature profiles as shown in FIG. 8 and given in Table 10. After the temperature experiments the solids are obtained by evaporation of the solvent at room temperature in a vacuum chamber.

TABLE 9
Well plate preparation values for high throughput crystallization of E7974.

| | A to F | | G to L | |
|---|---|---|---|---|
| Stock | 20 μL | | 40 μL | |
| volume | (Concentration | | (Concentration stock | |
| | stock solution | | solution 100 mg/ml) | |
| | 100 mg/ml) | | | |
| Starting | 2.0 | mg | 4.0 | mg |
| material | | | | |
| mass | | | | |
| Solvent | 40 | μL | 40 | μL |
| volume | | | | |
| Concentration | 50 | mg/ml | 100 | mg/ml |

TABLE 10
Temperature profiles used for the 12 plates.

| | Heating rate | Tᵢₙᵢₜᵢₐₗ | Hold | Cooling rate | T_{final} | Hold |
|---|---|---|---|---|---|---|
| Plate | (° C./min) | (° C.) | (min) | (° C./h) | (° C.) | (hours) |
| 1 | 4.8 | 75 | 30 | 1 | 5 | 1 |
| 2 | 4.8 | 75 | 30 | 5 | 5 | 1 |
| 3 | 4.8 | 75 | 30 | 30 | 5 | 1 |
| 4 | 4.8 | 75 | 30 | 1 | 5 | 72 |
| 5 | 4.8 | 75 | 30 | 5 | 5 | 72 |
| 6 | 4.8 | 75 | 30 | 30 | 5 | 72 |
| 7 | 4.8 | 75 | 30 | 1 | 25 | 1 |
| 8 | 4.8 | 75 | 30 | 5 | 25 | 1 |
| 9 | 4.8 | 75 | 30 | 30 | 25 | 1 |
| 10 | 4.8 | 75 | 30 | 1 | 25 | 72 |
| 11 | 4.8 | 75 | 30 | 5 | 25 | 72 |
| 12 | 4.8 | 75 | 30 | 30 | 25 | 72 |

Table 11 lists the solvents which gave crystalline forms of E7974 in the high throughput crystalization studies. The crystalline forms could generally be observed by visual inspection but was determined by powder X-ray diffraction (PXRD). PXRD analysis also showed amorphous E7974 in some wells. The amorphous form is not reported here.

TABLE 11

| Form | Solvent |
|---|---|
| M₁ | acetone |
| O₁ | nitrobenzene; amyl ether; chlorobenzene; toluene; water/acetone (20:80); |
| | water/2-propanol (20:80); water/2-propanol (10:90); water/EtOH (10:90); |
| | water/Ethanol (20:80); 1-bromopropane; cyclohexanone; DMA; DMF; TBME; |
| | MIBK; 1,4-dioxane; 1-nitropropane; TFE; diisobutyl ketone; |
| | 2-butoxyethylacetate; trifluoromethyl toluene; chloroform; diisopropylether; |
| | isophorone; n-butylacetate; THF; nitrobenzene; trifluomethane toluene |
| M₂ | 1,4-dioxane; THF; water/acetone; MIBK; 1-bromopropane; 1-nitropropane; |
| | 2-butoxyethyl acetate; acetone; acetonitrile; amyl ether; chloroform, DCM; |
| | DMF; ethylacetate/n-heptane (50:50); ethylacetate; n-butylacetate; |
| | nitromethane; trichloroethylene; water/acetone (20:80); water/acetone (10:90); water |
| | /acetonitrile (10:90); water/Ethanol (20:80) |

High Throughput PXRD Analysis of Crystalline Forms

After the crystallization experiments and the solvent evaporation the crystalline products were harvested. PXRD patterns were obtained using a high throughput PXRD set-up. The plates were mounted on a Bruker GADDS diffractometer that is equipped with a Hi-Star area detector. The PXRD platform is calibrated using Silver Behenate for the long d-spacings and Corundum for the short d-spacings. The data collection was carried out at room temperature using monochromatic CuK_{α} radiation in the region of 2Θ between 1.5 and 41.5°. The diffraction pattern of each well was collected in two 2Θ ranges (1.5≦2Θ≦21.5° for the 1st frame, and 19.5≦2Θ≦41.5° for the second frame) with an exposure time of 90 s for each frame. The carrier material used during PXRD analysis of most samples was transparent to X-rays and contributed only slightly to the background. No background subtraction or curve smoothing was applied to the PXRD patterns.

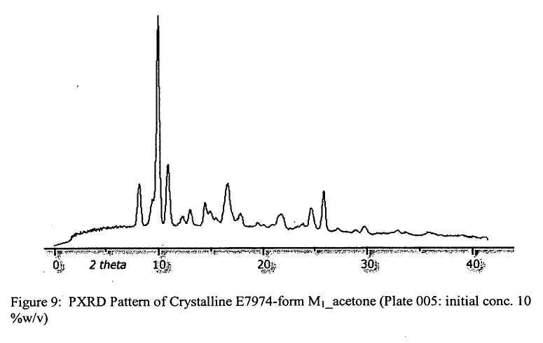

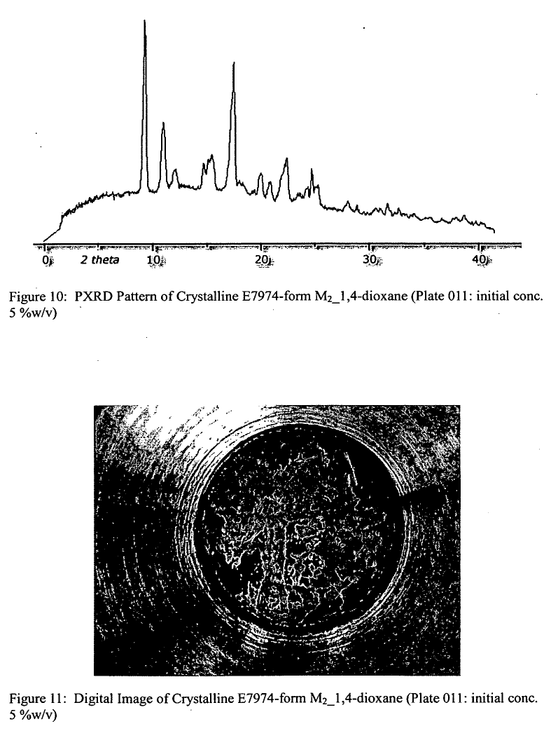

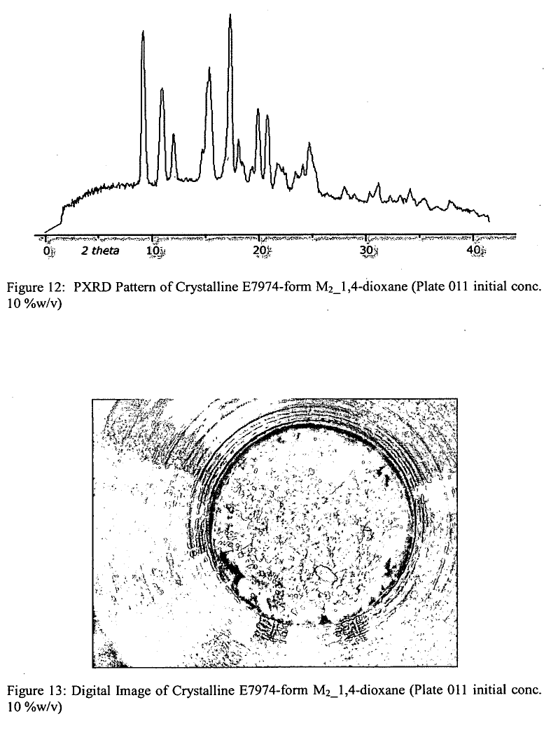

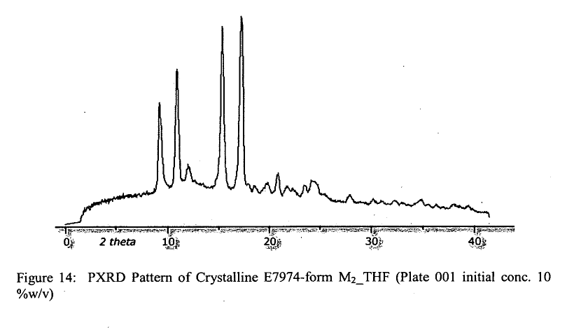

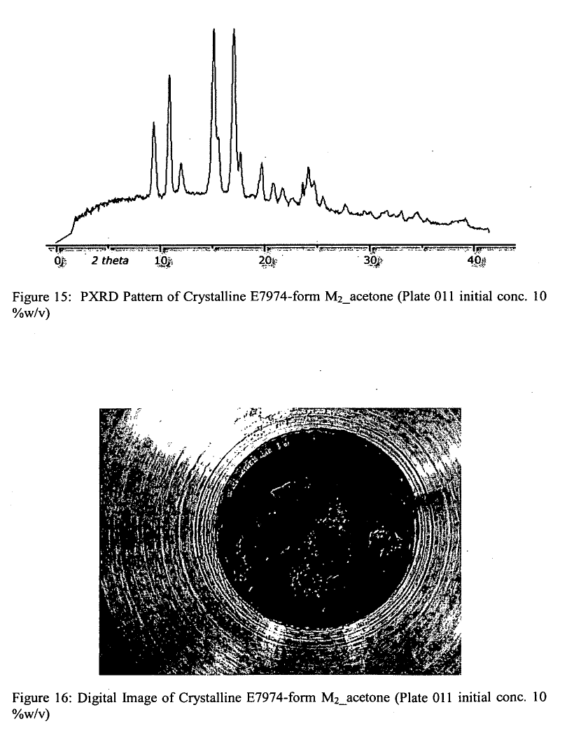

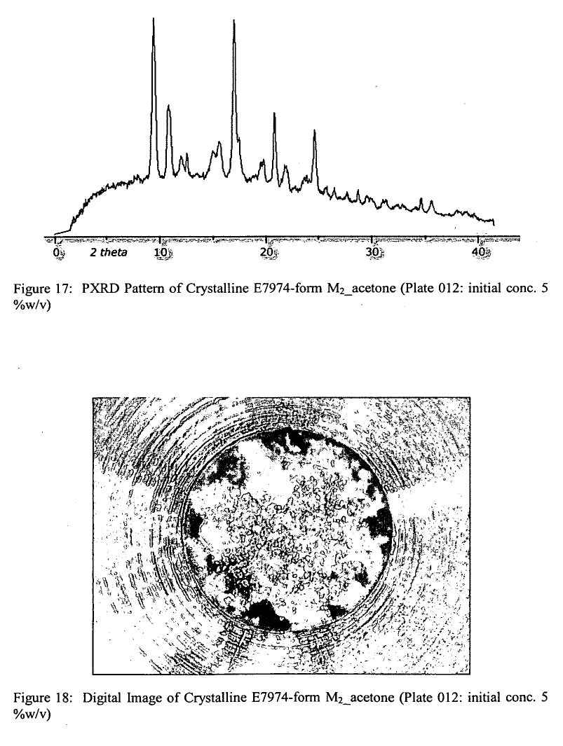

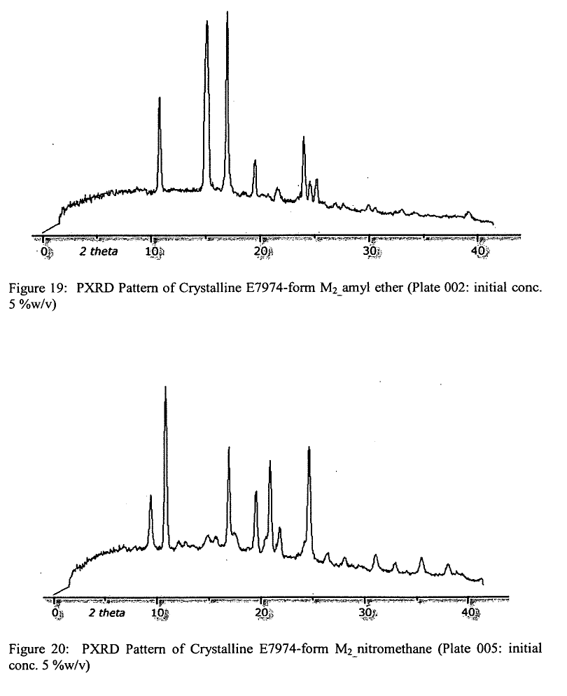

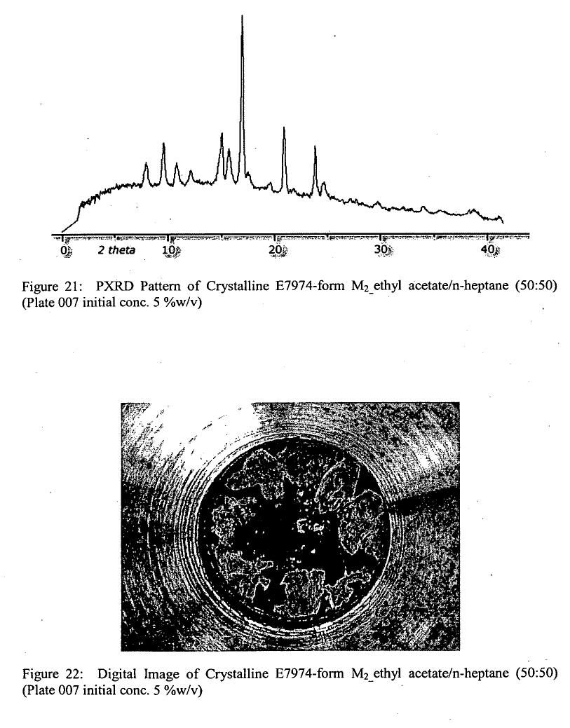

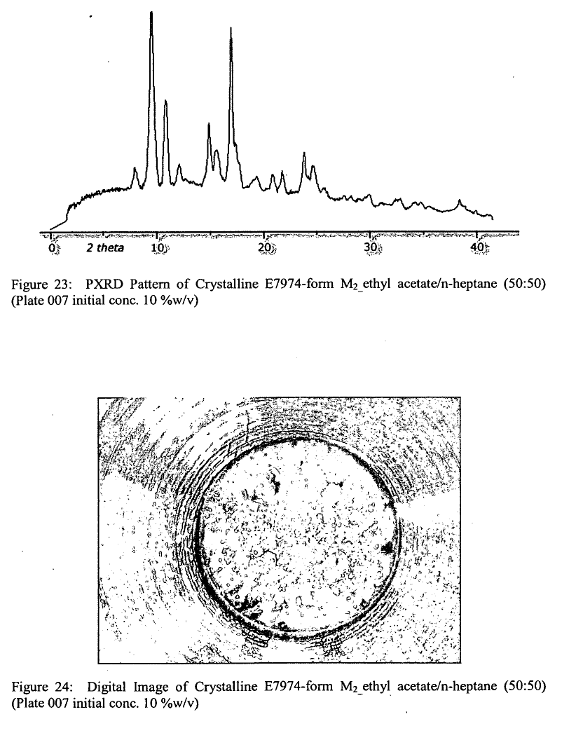

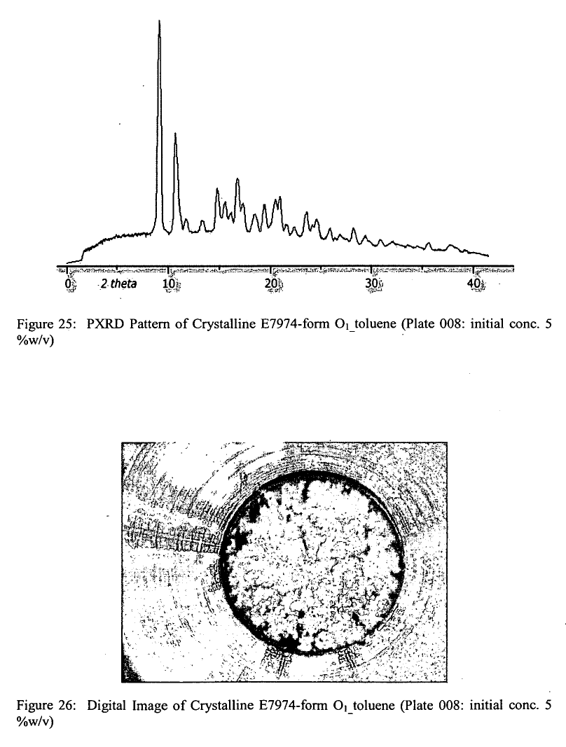

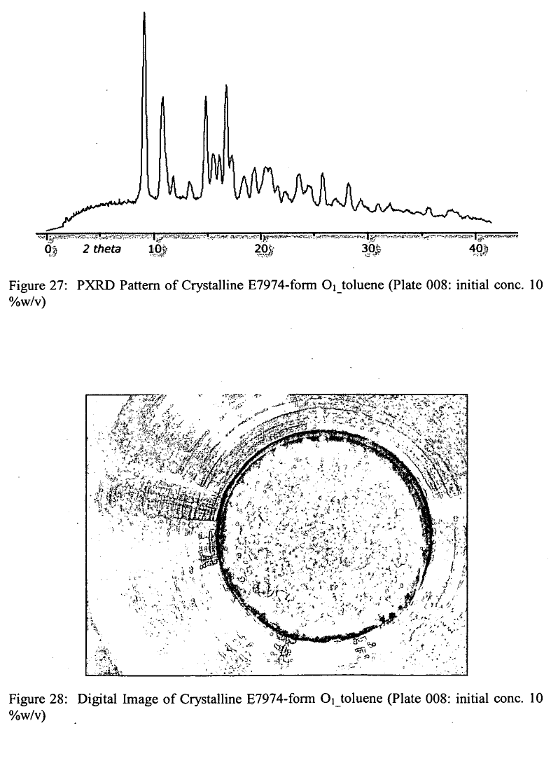

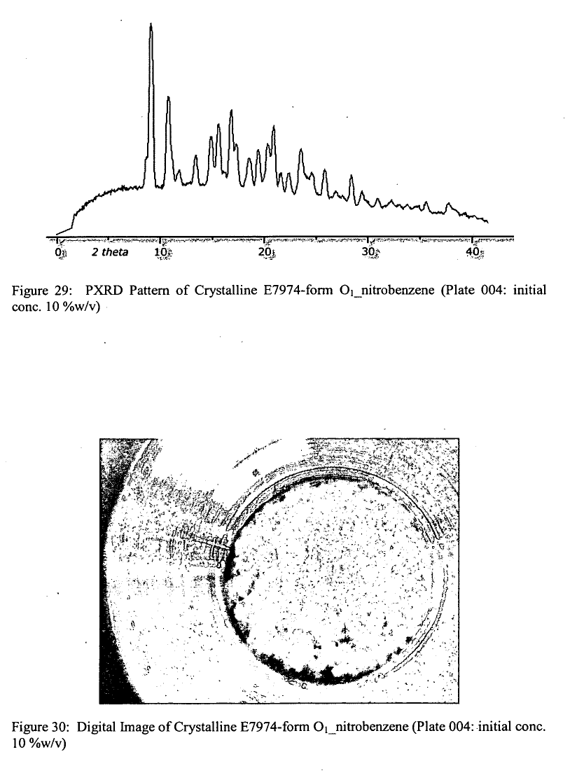

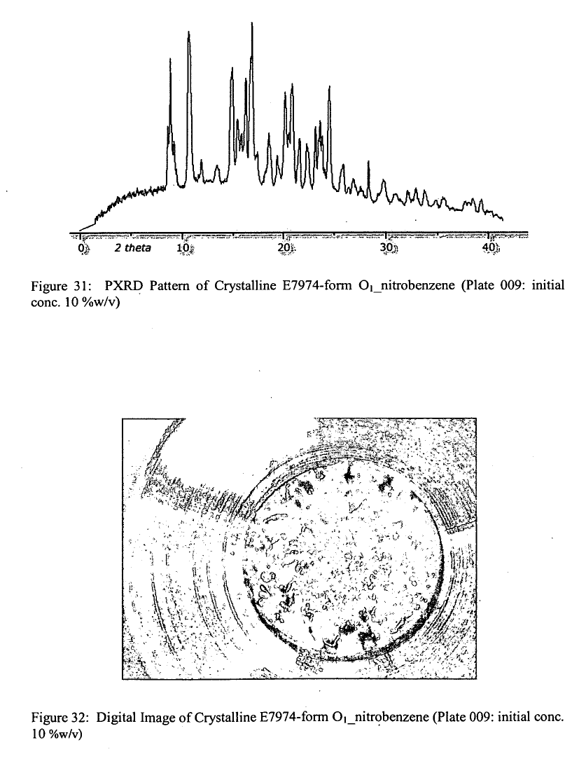

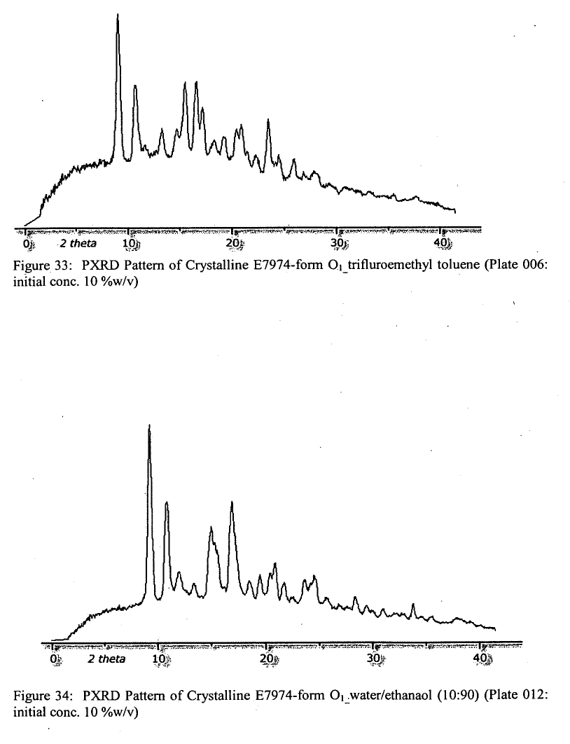

FIGS. 9-34 show PXRD patterns and digital images of various representative host-guest solvates of the M₁, M₂, and O₁ crystalline forms of E7974 identified in the high throughput crystallization studies. As can be seen from the Figures, the solvents occupying the cavity in the crystal structure do not significantly change the PXRD pattern of the host form. The PXRD patterns of the host-guest solvates may not be as sharp as those of the corresponding unsolvated host. The PXRD peaks may be broader or less intense depending on the solvent or concentration. The PXRD patterns of host-guest solvated forms, however, show the majority if not all of the characteristic peaks for the unsolvated host.

Single Crystal Structure Determination

Suitable single crystals from the high throughput studies were selected and glued to a glass fibre, which is mounted on a X-ray diffraction goniometer. X-ray diffraction data are collected for the mounted crystals at a temperature of 233 K using a KappaCCD system and MoKα radiation generated by a FR590 X-ray generator (Bruker Nonius, Delft, The Netherlands). Unit-cell parameters and crystal structure are determined and refined using the software package maXus (Mackay et al., 1997). From the crystal structure the theoretical X-ray powder diffraction pattern can be calculated using PowderCell for Windows version 2.3 (Kraus et al., 1999).

Single Crystal structure of form M₂_{_{—}}amyl ether

The crystal structure of crystalline form M₂_{_{—}}amyl ether was determined based on a single crystal obtained after the crystallization experiment with amyl ether (prepared according to the procedure of plate 002, low concentration, see Figure M₂_{_{—}}amyl ether). Table 12 presents a summary of the crystallographic data resulted from the crystal structure determination. The single-crystal results indicated that form M₂_{_{—}}amyl ether is a solvated form with amyl ether.

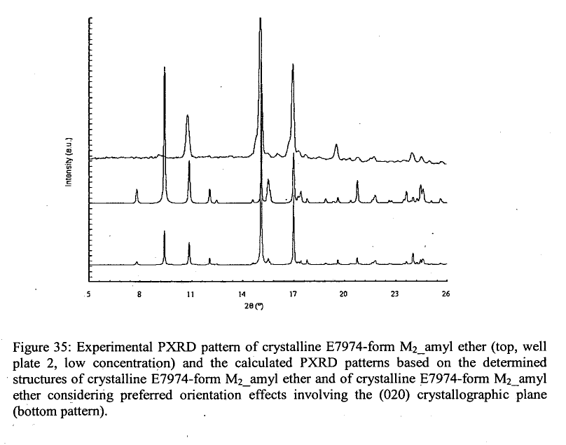

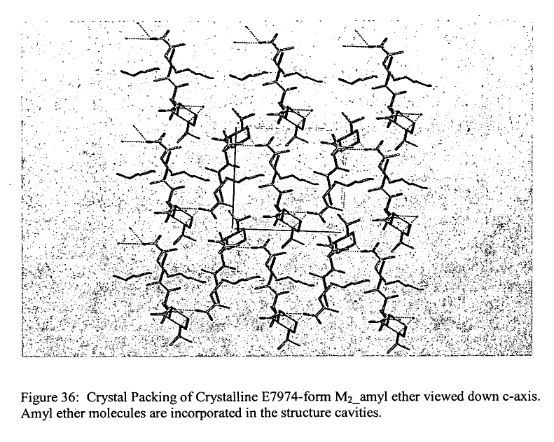

FIG. 35 presents a comparison of the experimental PXRD pattern with the calculated pattern based on the determined crystal structure of form M₂_{_{—}}amyl ether. The two PXRD patterns show differences, indicating that preferred orientation effects could be present in the bulk material and form M₂_{_{—}}amyl ether might be a single form. In order to confirm this, the preferred orientation (PO) was simulated assuming the (020) crystallographic plane as the PO plane in a March Dollase model (note that PO=1.0 represents no preferred orientation). The simulated PXRD pattern of form M₂_{_{—}}amyl ether considering the PO effects is similar to the experimental PXRD pattern of form M₂_{_{—}}amyl ether (see FIG. 35, the first and the third patterns from top), indicating that indeed PO effects are present in the bulk material and that the crystal structure of form M₂_{_{—}}amyl ether corresponds to the bulk material. FIG. 36 depicts the crystal packing of form M₂_{_{—}}amyl ether viewed down c-axis. Amyl ether molecules are incorporated in the structure cavities.

TABLE 12
Crystal Data (form M₂_{_{—}}amyl ether)

| Formula | 2(C24H43N3O4),2(C4)O |
|---|---|
| Formula Weight | 987.31 |
| Crystal System | Monoclinic |
| Space group | P21 (No. 4) |
| a, b, c [Angstrom] | 11.7440(7) 11.7610(7) 11.8140(10) |
| alpha, beta, gamma [deg] | 90 105.863(2) 90 |
| V [Ang**3] | 1569.62(19) |
| Z | 1 |
| D(calc) [g/cm**3] | 1.044 |
| F(000) | 536 |
| Data Collection | |
| Temperature (K) | 293 |
| Radiation [Angstrom] | MoKa .71073 |
| Theta Min-Max [Deg] | 2.8, 27.4 |
| Dataset | −15: 14; −13: 12; −15: 11 |
| Tot., Uniq. Data, R(int) | 7831, 6061, 0.045 |
| Observed data [I > 2.0 sigma(I)] | 3050 |
| Refinement | |
| Nref, Npar | 6061, 308 |
| R, wR2, S | 0.0812, 0.2171, 1.00 |

w = 1/[¥s\hat{ }2\hat{ }(Fo\hat{ }2\hat{ }) + (0.1000P)\hat{ }2\hat{ }] where P = (Fo\hat{ }2\hat{ } + 2Fc\hat{ }2\hat{ })/3

Crystal structure of crystalline E7974 form O₁_{_{—}}nitrobenzene

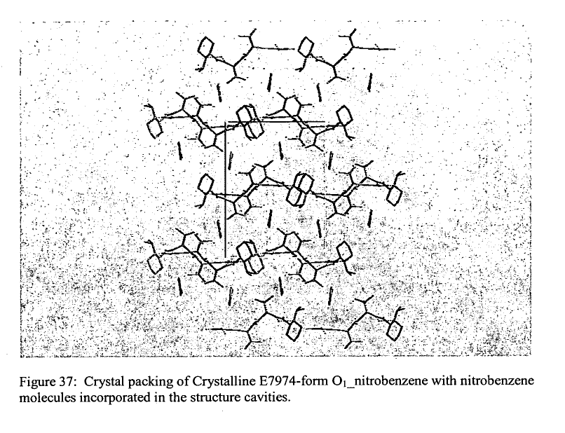

The crystal structure of crystalline E7974 form O1_nitrobenzene was determined from the single-crystal data collected from the material obtained after the crystallization experiment with nitrobenzene (prepared according to plate 003, high concentration, crystallization temperature 5° C.). The PXRD analysis indicated that the material was a mixture forms M₂_{_{—}}nitrobenzene and form O₁_{_{—}}nitrobenzene but a suitable single crystal of form O₁_{_{—}}nitrobenzene was be found in the mixture and analyzed. Table 13 presents a summary of the crystallographic data resulted from the crystal structure determination. FIG. 37 shows the crystal packing of form O₁_{_{—}}nitrobenzene with nitrobenzene molecules incorporated in the structure cavities. The single-crystal results indicated that the crystal is a solvated form with nitrobenzene with the nitrobenzene molecules are incorporated in the crystal structure cavities.

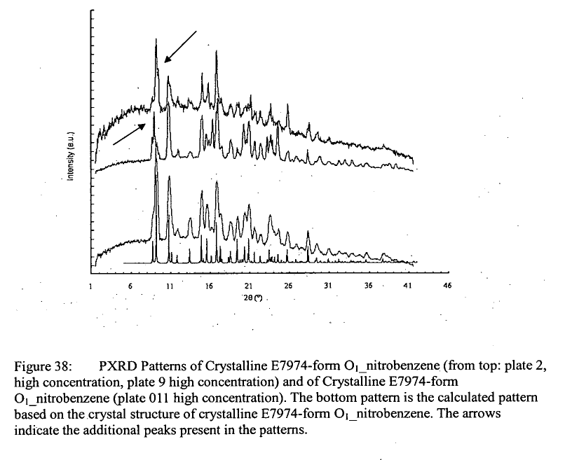

FIG. 38 presents a comparison of the experimental PXRD pattern with the calculated pattern based on the determined crystal structure of form O₁_{_{—}}nitrobenzene. The two PXRD patterns are highly similar, indicating that the crystal structure of form O₁_{_{—}}nitrobenzene is representative for the bulk material as a single crystalline form.

TABLE 13
Crystal Data (form O₁_{_{—}} nitrobenzene)

| Formula | C24 H43 N3 O4, C6 H5 N O2 |
|---|---|
| Formula Weight | 560.72 |
| Crystal System | Orthorhombic |
| Space group | P212121 (No. 19) |
| a, b, c [Angstrom] | 11.9490(7), 14.0820(8), 19.3760(14) |
| V [Ang**3] | 3260.3(4) |
| Z | 4 |
| D(calc) [g/cm**3] | 1.142 |
| Mu(MoKa) [/mm] | 0.080 |
| F(000) | 1216 |
| Data Collection | |
| Temperature (K) | 293 |
| Radiation [Angstrom] | MoKa 0.71073 |
| Theta Min-Max [Deg] | 2.2, 23.7 |
| Dataset | −12: 12; −13: 13; −20: 19 |
| Tot., Uniq. Data, R(int) | 2501, 2501, 0.000 |
| Observed data [I > 2.0 sigma(I)] | 1329 |
| Refinement | |
| Nref, Npar | 2501, 309 |
| R, wR2, S | 0.0970, 0.2640, 1.10 |

w = 1/[¥s\hat{ }2\hat{ }(Fo\hat{ }2\hat{ }) + (0.1156P)\hat{ }2\hat{ } + 1.4558P]
where P = (Fo\hat{ }2\hat{ } + 2Fc\hat{ }2\hat{ })/3

| | |
|---|---|
| Max. and Av. Shift/Error | 0.02, 0.00 |
| Mm. and Max. Resd. Dens. | −0.25, 0.24 |
| [e/Ang\hat{ }3] | |

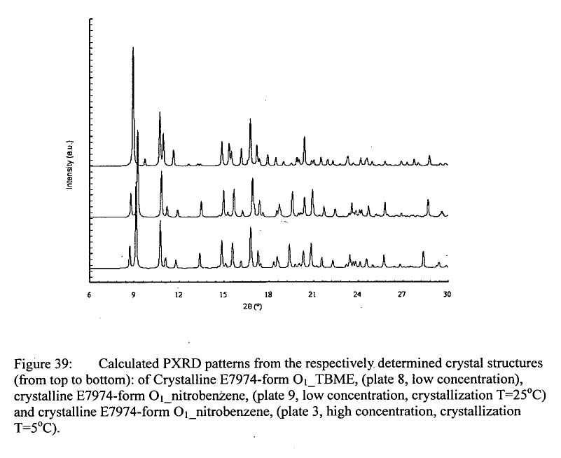

Single crystal analyses were also performed for O₁ forms obtained from nitrobenzene at 25° C. crystallization temperature and host-guest solvated form O₁ obtained from TBME. FIG. 39 presents the comparison of the calculated patterns based on the determined structures. It can be concluded that different crystallization conditions lead to small variations in the unit cell parameters of form O₁_{_{—}}nitrobenzene (see FIG. 39, patterns 2 and 3 from top; small shifts in the peaks positions are present). These variations in the unit cell parameters could be explained by the difference in the degree of disorder present in the crystal structures (a higher disorder degree was found in case of form O₁_{_{—}}solvent crystallized at 25° C. than at 5° C.). On the other hand a different solvent embedded in the crystal structure resulted in more significant variations of the unit cell parameters and lead additionally to modifications of the diffraction intensities (see FIG. 39, pattern 1 from top compared to the other patterns). For example, a third host-guest form O₁_{_{—}}-solvate was obtained from trifluoromethyl toluene.

### Example 9

### Characterization of Crystalline, Host-Guest Solvate E7974-Form M₁_{_{—}}Acetonitrile by Powder X-Ray Diffraction (PXRD) and Infrared (IR) Spectroscopy

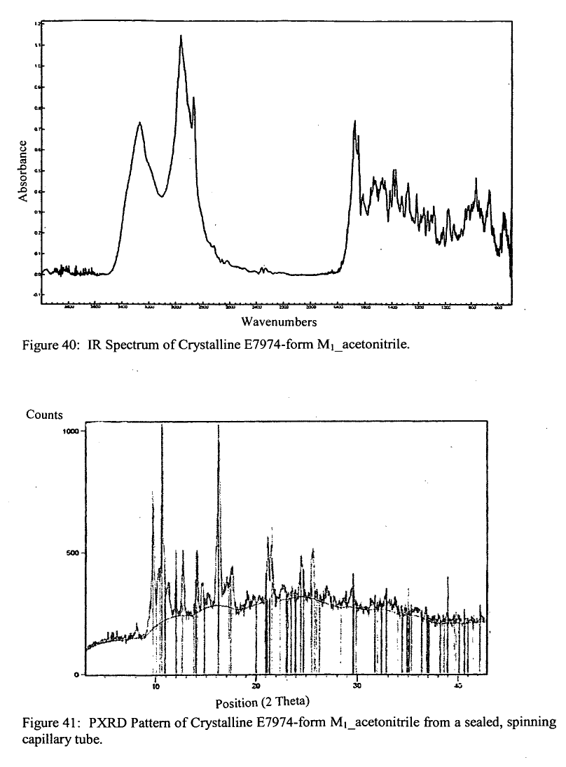

FIG. 40 shows the IR spectram of crystalline, host-guest solvated crystalline E7974-form M₁_{_{—}}acetonitrile. The IR spectram was obtained using a technique as described in Example 3.

FIG. 41 depicts the PXRD pattern of crystalline E7974 form M₁_{_{—}}acetonitrile from a sealed, spinning capillary tube. PXRD data were collected at ambient temperature on a PANalytical X'Pert Pro Θ/Θ diffractometer (00008819), operating with copper radiation at 45 kV and 40 mA, using a X'Celerator detector (00008823). The PXRD unit is equipped with a capillary spinner stage and a standard PC with Windows XP® operating system and PANalytical X'Pert Data Collector v 2.1a. Each stage was aligned upon installation using NBS silicon powder as a standard. Table 14 identifies the peaks in the PXRD pattern in FIG. 41. Table 15 lists preferred characteristic peaks in the PXRD pattern of form M₁_{_{—}}acetonitrile any three or more of which should sufficiently identify crystalline E7974 form M₁_{_{—}}acetonitrile any four or more of which should sufficiently identify crystalline E7974 form M₁_{_{—}}acetonitrile.

TABLE 14

| | Rel. Int. |
|---|---|
| Pos. [° 2Th.] | [%] |
| 8.1 | 5.54 |
| 9.7 | 60.63 |
| 10.3 | 28.78 |
| 10.6 | 100 |
| 11.3 | 19.89 |
| 12.0 | 21.02 |
| 12.7 | 37.43 |
| 14.1 | 32.26 |
| 14.6 | 13.67 |
| 15.2 | 7.7 |
| 16.2 | 91.26 |
| 17.6 | 19.68 |
| 19.1 | 6.01 |
| 19.8 | 3.92 |
| 21.2 | 24.86 |
| 21.6 | 29.69 |
| 22.7 | 7.36 |
| 24.6 | 19.21 |
| 25.5 | 20.1 |
| 25.7 | 26.64 |
| 27.0 | 6.86 |
| 29.4 | 8.52 |
| 31.3 | 3.94 |
| 36.0 | 3.35 |
| 38.1 | 2.7 |
| 38.8 | 2.67 |

TABLE 15
Preferred Characteristic PXRD Peaks
of Crystalline E7974-form
M₁_{_{—}}_{acetonitrile.}
2 theta (degree)
9.7 ± 0.2
10.6 ± 0.2
11.3 ± 0.2
12.0 ± 0.2
12.7 ± 0.2
14.1 ± 0.2
14.6 ± 0.2
16.2 ± 0.2
17.6 ± 0.2
21.2 ± 0.2
21.6 ± 0.2

### Example 10

Characterization of Crystalline, Host-guest Solvate E7974-form M₂_{_{—}}1,4 dioxane by Powder X-ray Diffraction (PXRD), Infrared (IR) Spectroscopy, and DSC. The PXRD pattern and IR spectrum were acquired using the techniques and equipment described in Examples 9 and 3, respectively. The DSC data was acquired using the procedure described in Example 4 but with a 2.15 g sample.

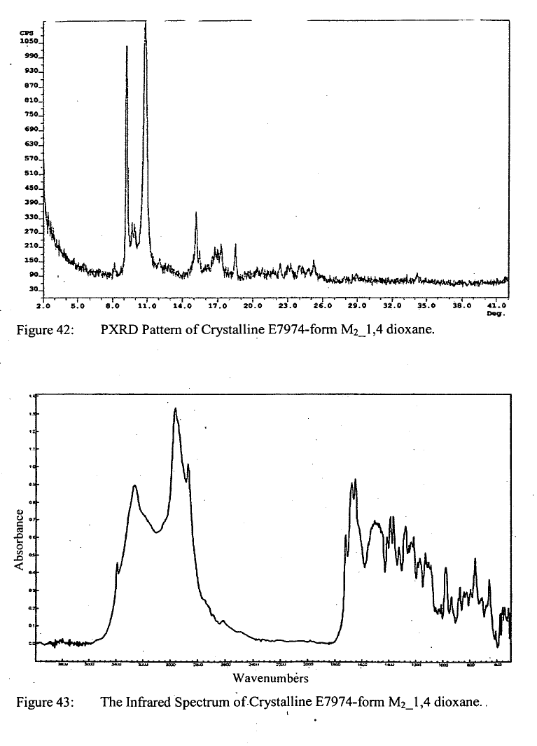

FIG. 42 shows the PXRD pattern of crystalline E7974-form M₂_{_{—}}1,4 dioxane host-guest solvate. Table 16 identifies the peaks in the PXRD pattern in FIG. 42. In this and the other PXRD patterns presented, some of the less intense reported peaks may not correspond to real peaks. Table 17 lists some characteristic peaks for crystalline E7974 form M₂_{_{—}}1,4 dioxane any three or more of which should sufficiently identify crystalline E7974 M₂_{_{—}}1,4 dioxane.

TABLE 16

| Peak Position | Relative |
|---|---|
| Deg. 2Θ ± 0.2 | Intensity |
| 8.1 | 25.45 |
| 9.2 | 619.73 |
| 9.7 | 123.67 |
| 9.9 | 124.75 |
| 10.8 | 680 |
| 11.5 | 55 |
| 12.0 | 40 |
| 12.5 | 25.83 |
| 12.7 | 23.52 |
| 13.0 | 23.33 |
| 15.2 | 160.65 |
| 15.5 | 61.37 |
| 15.9 | 22.47 |
| 16.1 | 28.12 |
| 16.2 | 30.67 |
| 16.5 | 48.4 |
| 16.8 | 64.58 |
| 16.9 | 65 |
| 17.0 | 67.5 |
| 17.3 | 84.42 |
| 18.5 | 84.32 |
| 20.3 | 22.47 |
| 20.8 | 29.23 |
| 21.6 | 21.73 |
| 22.3 | 37.7 |
| 23.0 | 30.5 |
| 23.2 | 32.77 |
| 24.0 | 33.85 |
| 24.2 | 36.73 |
| 24.7 | 23.93 |
| 24.8 | 25.32 |
| 25.2 | 49.23 |
| 28.9 | 23.17 |

TABLE 17
Characteristic PXRD Peaks of
Crystalline E7974-form M₂_{_{—}}1,4
dioxane.
2 theta (degree)¹
9.2 ± 0.3
10.8 ± 0.3
15.2 ± 0.3
18.5 ± 0.3
¹A larger degree of uncertainty is given due to the breadth of the peaks.

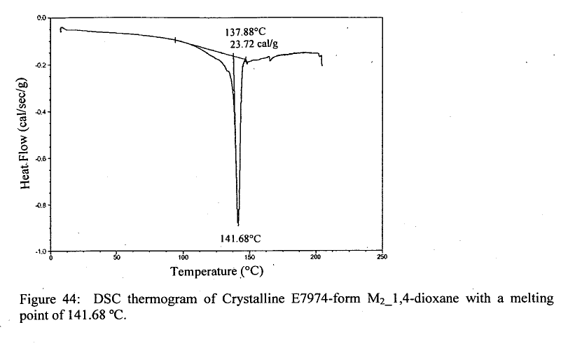

The infrared spectrum of crystalline E7974-form M₂_{_{—}}1,4 dioxane is shown in FIG. 43. FIG. 44 shows the DSC thermogram of crystalline E7974-form M₂_{_{—}}1,4-dioxane with a melting point of 141.68° C.

### Example 11

### Characterization of Crystalline E7974-Form O₁_{_{—}}Unsolvated by Powder X-Ray Diffraction (PXRD), Infrared (IR) Spectroscopy, DSC, and ¹³C CP/MAS NMR

The PXRD pattern, and IR spectrum data was acquired using the techniques and equipment described in Examples 9 and 3, respectively. The ¹³C CP/MAS NMR spectrum was obtained as described in Example 5. The DSC data was acquired using the procedure described in Example 4 using a 1.79 g sample.

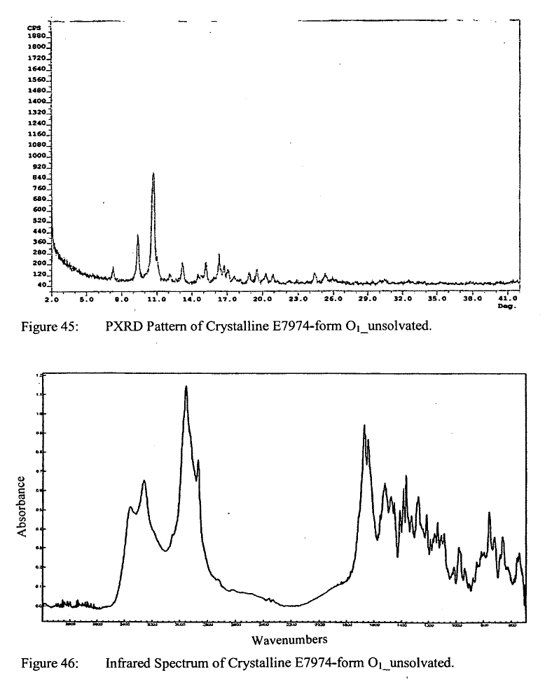

FIG. 45 shows the PXRD pattern of crystalline E7974-form O₁_{_{—}}unsolvated. Table 18 identifies the peaks in the PXRD pattern in FIG. 45. Table 19 lists some preferred characteristic peaks for crystalline E7974 form O₁_{_{—}}unsolvated any three or more of which should sufficiently identify crystalline E7974 form O₁_{_{—}}unsolvated.

TABLE 18

| Peak Position | Relative |
|---|---|
| Deg. 2Θ ± 0.2 | Intensity |
| 7.3 | 45.63 |
| 9.4 | 219.52 |
| 10.1 | 35.83 |
| 10.7 | 535.77 |
| 11.0 | 111.83 |
| 12.1 | 31.75 |
| 13.2 | 88.92 |
| 14.5 | 32.67 |
| 14.8 | 28.18 |
| 15.2 | 95.57 |
| 16.3 | 112.33 |
| 16.7 | 81.27 |
| 17.0 | 60.25 |
| 17.5 | 26.28 |
| 18.8 | 48.78 |
| 19.5 | 66.35 |
| 20.2 | 40.7 |
| 20.9 | 35.07 |
| 24.3 | 50.83 |
| 25.3 | 47.12 |
| 25.9 | 24.12 |
| 30.4 | 22.77 |

```
TABLE 19
Preferred Characteristic PXRD Peaks
of Crystalline E7974-form
O₁_{_{—}}_{unsolvated.}
2 theta (degree)
  7.3 ± 0.2
  9.4 ± 0.2
10.7 ± 0.2
13.2 ± 0.2
15.2 ± 0.2
```

Crystalline E7974 form O₁_{_{—}}unsolvated is preferably characterized by having at least three peaks in its powder X-ray diffraction pattern selected from the group consisting of 7.3±0.2Θ, 9.4±0.2Θ, 10.7±0.2Θ, 12.1±0.2Θ, and 15.2±0.2Θ.

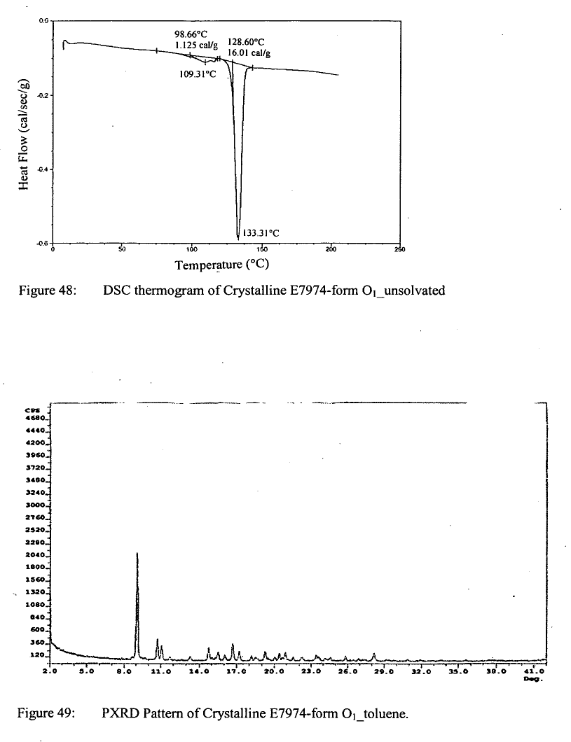

The infrared spectrum of crystalline E7974-form O₁_{_{—}}unsolvated is shown in FIG. 46. FIG. 48 shows the DSC thermogram of crystalline E7974-form M₂_{_{—}}O₁_{_{—}}unsolvated with a melting point of 133.31° C.

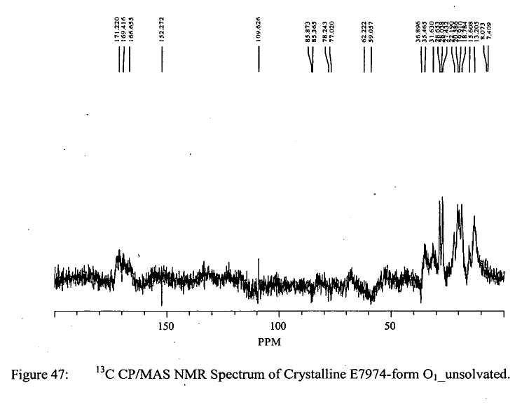

FIG. 47 shows the resulting ¹³C CP/MAS NMR spectrum of crystalline E7974-form O₁_{_{—}}unsolvated. The quality of this spectrum is not as good as that of the crystalline E7974-form M₁_{_{—}}unsolvated spectrum. While the exact reason why the quality of the spectrum is not very high is unknown, it may be related to particle size issues and/or crystal quality in the particular sample. Chemical shifts are reported to be within ±0.3 ppm.

### Example 12

### Characterization of Crystalline, Host-Guest Solvate E7974-Form O₁_{_{—}}Toluene by Powder X-Ray Diffraction (PXRD), Infrared (IR) Spectroscopy, and DSC

The PXRD pattern and IR spectrum were acquired using the techniques and equipment described in Example 3. The DSC data was acquired using the procedure described in Example 4 using a 3.75 g sample.

FIG. 49 shows the PXRD pattern of crystalline E7974-form O₁_{_{—}}toluene host-guest solvate. Table 20 identifies the peaks in the PXRD pattern in FIG. 49. Table 21 lists some preferred characteristic peaks for crystalline E7974 form O₁_{_{—}}toluene any three or more of which should sufficiently identify crystalline E7974 form O₁_{_{—}}toluene.

TABLE 20

| Peak Position | Relative |
|---|---|
| Deg. 2Θ ± 0.2 | Intensity |
| 8.7 | 26.43 |
| 9.0 | 1392.53 |
| 10.7 | 268.37 |
| 11.0 | 177.82 |
| 11.7 | 40.28 |
| 13.3 | 42.17 |
| 14.8 | 158.22 |
| 15.0 | 34.65 |
| 15.3 | 40.53 |
| 15.5 | 96.8 |
| 16.0 | 71.1 |
| 16.7 | 222.67 |
| 17.2 | 121.78 |
| 18.2 | 51.17 |
| 18.5 | 40.17 |
| 19.2 | 110.03 |
| 19.5 | 35.68 |
| 20.0 | 39.17 |
| 20.4 | 98.9 |
| 20.6 | 34.87 |
| 20.9 | 104.2 |
| 21.5 | 48.15 |
| 22.2 | 37.88 |
| 23.4 | 73.47 |
| 23.6 | 41.08 |
| 24.1 | 34.42 |
| 24.5 | 41.62 |
| 25.8 | 61.92 |
| 26.9 | 25.95 |
| 28.1 | 106.58 |

TABLE 21
Preferred Characteristic PXRD Peaks
of Crystalline E7974-form
O₁_{_{—}}_{toluene.}
2 theta (degree)
9.0 ± 0.2
10.7 ± 0.2
11.0 ± 0.2
14.8 ± 0.2
16.7 ± 0.2
17.2 ± 0.2

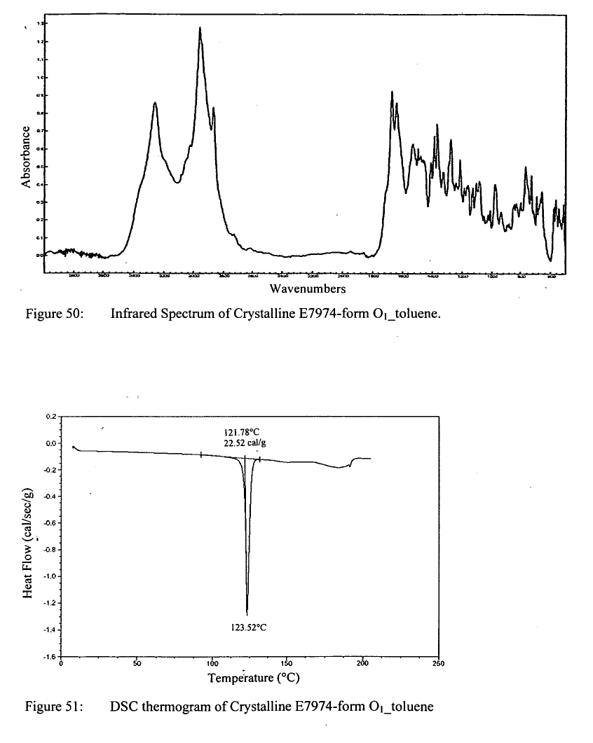

The infrared spectrum of crystalline E7974-form O_{—}_{_{—}}toluene is shown in FIG. 50. FIG. 51 shows the DSC thermogram of crystalline E7974-form O₁_{_{—}}toluene with a melting point of 123.52° C.

The claimed invention is:

1. Crystalline (2E,4S)-4-[(N-{[(2R)-1-isopropylpiperidin-2-yl]-carbonyl}-3-methyl-L-valyl)(methyl)amino]-2,5-dimethylhex-2-enoic acid.

2. A pharmaceutical composition comprising crystalline (2E,4S)-4-[(N-{[(2R)-1-isopropylpiperidin-2-yl]-carbonyl}-3-methyl-L-valyl)(methyl)amino]-2,5-dimethylhex-2-enoic acid and a pharmaceutically acceptable carrier.

3. A crystalline form of (2E,4S)-4-[(N-{[(2R)-1-isopropylpiperidin-2-yl]-carbonyl}-3-methyl-L-valyl)(methyl)amino]-2,5-dimethylhex-2-enoic acid comprising crystalline (2E,4S)-4-[(N-{[(2R)-1-isopropylpiperidin-2-yl]-carbonyl}-3-methyl-L-valyl)(methyl)amino]-2,5-dimethylhex-2-enoic acid and a host-guest amount of solvent within a cavity of the crystal lattice.

4. Crystalline (2E,4S)-4-[(N-{[(2R)-1-isopropylpiperidin-2-yl]-carbonyl}-3-methyl-L-valyl)(methyl)amino]-2,5-dimethylhex-2-enoic acid according to claim 3, wherein the solvent is a pharmaceutically acceptable solvent.

5. A monoclinic crystalline form, M₁, of (2E,4S)-4-[(N-{[(2R)-1-isopropylpiperidin-2-yl]-carbonyl}-3-methyl-L-valyl)(methyl)amino]-2,5-dimethylhex-2-enoic acid, characterized by having at least four peaks in its powder X-ray diffraction pattern selected from the group consisting of 8.2±0.2Θ, 10.0±0.2Θ, 10.9±0.2Θ, 13.0±0.2Θ, 14.3±0.2Θ, 16.3±0.2Θ, and 17.9±0.2Θ.

6. A monoclinic, host-guest soluated crystalline form, M₁, of (2E,4S)-4-[(N-{[(2R)-1-isopropylpiperidin-2-yl]-carbonyl}-3-methyl-L-valyl)(methyl)amino]-2,5-dimethylhex-2-enoic acid, comprising crystalline (2E,4S)-4-[(N-{[(2R)-1-isopropylpiperidin-2-yl]-carbonyl}-3-methyl-L-valyl)(methyl)amino]-2,5-dimethylhex-2-enoic acid according to claim 5 and a host-guest amount of an organic solvent within a cavity of the crystal lattice.

7. A monoclinic, host-guest crystal form, M₁, according to claim 6, wherein the organic solvent is selected from the group consisting of acetone and acetonitrile.

8. A monoclinic crystalline form, M₁, of (2E,4S)-4-[(N-{[(2R)-1-isopropylpiperidin-2-yl]-carbonyl}-3-methyl-L-valyl)(methyl)amino]-2,5-dimethylhex-2-enoic acid, characterized by a 13C CP/MAS spectrum having at least three peaks selected from 14.1±0.3 ppm, 15.3±0.3 ppm, 19.1±0.3 ppm, 21.3±0.3 ppm, 23.7±0.3 ppm, and 27.2±0.3 ppm.

9. A monoclinic, host-guest crystalline form, M₁, of (2E,4S)-4-[(N-{[(2R)-1-isopropylpiperidin-2-yl]-carbonyl}-3-methyl-L-valyl)(methyl)amino]-2,5-dimethylhex-2-enoic acid, comprising crystalline (2E,4S)-4-[(N-{[(2R)-1-isopropylpiperidin-2-yl]-carbonyl}-3-methyl-L-valyl)(methyl)amino]-2,5-dimethylhex-2-enoic acid according to claim 8 and an organic solvent within a cavity of the crystal lattice.

10. A monoclinic crystal form, M₁, according to claim 6, wherein the organic solvent is selected from the group consisting of acetone and acetonitrile.

11. A solvated monoclinic crystalline form, M₂, of (2E,4S)-4-[(N-{[(2R)-1-isopropylpiperidin-2-yl]-carbonyl}-3-methyl-L-valyl)(methyl)amino]-2,5-dimethylhex-2-enoic acid comprising crystalline (2E,4S)-4-[(N-{[(2R)-1-isopropylpiperidin-2-yl]-carbonyl}-3-methyl-L-valyl)(methyl)amino]-2,5-dimethylhex-2-enoic acid and a host-guest amount of solvent within a cavity of the crystal lattice, characterized by having at least three peaks in its powder X-ray diffraction pattern selected from the group consisting of 9.2±0.2Θ, 10.8±0.2Θ, 15.2±0.2Θ, 16.9±0.2Θ, and 18.5±0.2Θ.

12. A solvated monoclinic crystal form, M₂, according to claim 11, wherein the organic solvent is selected from the group consisting of 1,4-dioxane, ethylacetate/n-heptane (50:50), acetone, and nitromethane.

13. A orthorhombic crystalline form, O₁, of (2E,4S)-4-[(N-{[(2R)-1-isopropylpiperidin-2-yl]-carbonyl}-3-methyl-L-valyl)(methyl)amino]-2,5-dimethylhex-2-enoic acid, characterized by having at least three peaks in its powder X-ray diffraction pattern selected from the group consisting of 7.3±0.2Θ, 9.4±0.2Θ, 10.7±0.2Θ, 13.2±0.2Θ, and 15.2±0.2Θ.

14. A solvated orthorhombic crystalline form, O₁, of (2E,4S)-4-[(N-{[(2R)-1-isopropylpiperidin-2-yl]-carbonyl}-3-methyl-L-valyl)(methyl)amino]-2,5-dimethylhex-2-enoic acid, comprising crystalline (2E,4S)-4-[(N-{[(2R)-1-isopropylpiperidin-2-yl]-carbonyl}-3-methyl-L-valyl)(methyl)amino]-2,5-dimethylhex-2-enoic acid and a host-guest amount of solvent within a cavity of the crystal lattice.

15. A solvated orthorhombic crystal form, O₁, according to claim 14, wherein the organic solvent is selected from the group consisting of toluene, water/ethanol (10:90), TBME, and nitrobenzene.

16. A pharmaceutical composition comprising a crystalline form of (2E,4S)-4-[(N-{[(2R)-1-isopropylpiperidin-2-yl]-carbonyl}-3-methyl-L-valyl)(methyl)amino]-2,5-dimethylhex-2-enoic acid as recited in claim 1, 3, 5, 6, 8, 9, 11, 13, or 14, and a pharmaceutically acceptable carrier.

17. A method for treating a proliferative disorder in a patient, comprising the step of administering to a patient in need thereof a composition comprising a crystalline form of (2E,4S)-4-[(N-{[(2R)-1-isopropylpiperiden-2-yl]-carbonyl}-3-methyl-L-valyl)(methyl)amino]-2,5-dimethylhex-2-enoic acid.

18. A method of claim 17, wherein the crystalline form is selected from the forms recited in claim 1, 3, 5, 6, 8, 9, 11, 13, or 14.

19. A method of claim 17, wherein the proliferative disorder is cancer.

20. A method of claim 19, wherein the cancer is selected from colorectal cancer, glioblastoma multiforme, breast cancer, prostate cancer, non-small cell lung cancer, hepatocellular, and esophageal/gastric cancer.

21. A method of claim 19, wherein the cancer is a taxane-resistant tumor.
